(19) United States
(12) Patent Application Publication
(10) Pub. No.: US 2026/0338769 A1
(43) Pub. Date: Oct. 29, 2026

(54) DETECTION OF NATURAL ANTIBODY REPERTOIRE VIA DETECTION OF REACTIVITY WITH ADDUCTS

(71) Applicant: The Trustees of Columbia University in the City of New York, New York, NY (US)

(72) Inventors: Shunya MASHIKO, New York, NY (US); Emmanuel ZORN, New York, NY (US)

(21) Appl. No.: 19/110,847

(22) PCT Filed: Sep. 11, 2023
(86) PCT No.: PCT/US2023/073888

§ 371 Date: Mar. 12, 2025

Related U.S. Application Data
(60) Provisional application No. 63/406,024, filed on Sep. 13, 2022.
(60) Provisional application No. 63/495,995, filed on Apr. 13, 2023.

(51) Int. Cl. G01N 33/543; G01N 33/50; G01N 33/53; G01N 33/575; G01N 33/57525; G01N 33/68

(52) U.S. Cl. CPC G01N 33/54313; G01N 33/5091; G01N 33/5308; G01N 33/57525; G01N 33/5758; G01N 33/6854

(57) ABSTRACT

Natural antibodies are an integral part of innate humoral immunity yet their development and polyreactive nature are still enigmatic. Here it is shown that characteristic monoclonal natural antibodies recognize common chemical moieties or adducts, supporting the view that polyreactive antibodies may often correspond to anti-adduct antibodies. Moreover, the development of IgM and IgG to 81 ubiquitous adducts from birth to old age was examined. Newborn IgM only reacted to a limited number of consensus determinants. This highly restricted neonatal repertoire abruptly diversified around 6 months of age through the development of antibodies to environmental antigens as well as age-driven epigenetic modifications. In contrast, the IgG repertoire was diverse across the entire lifespan. The studies set forth reveal an unrecognized but significant component of humoral immunity directed to common adducts.

### CROSS-REFERENCE TO RELATED APPLICATIONS

This application claims the benefit of U.S. Provisional Application Ser. No. 63/406,024, filed Sep. 13, 2022; and claims benefit of U.S. Provisional Application Ser. No. 63/495,995, filed Apr. 13, 2023. The entire contents of this application is hereby incorporated by reference as if fully set herein.

### GOVERNMENT SUPPORT CLAUSE

This invention was made with government support under AI131339 awarded by the National Institutes of Health. The government has certain rights in the invention.

### BACKGROUND

Natural antibodies (Nabs) play an essential part in protective humoral immunity and are commonly regarded as a first line of defense against pathogenic bacteria and viruses^{1, 2, 3}. In addition to their protective capabilities, Nabs also have crucial homeostatic functions associated with their capacity to bind self-determinants^{4, 5}. For instance, Nabs reactive to apoptotic cells promote efferocytosis and clearance of cellular debris^{6, 7, 8}. Disruption of these important functions can have important consequences as exemplified by the increase incidence of allergies or autoimmune diseases in patients with primary antibody deficiencies⁹.

Despite an abundant literature, Nabs are still loosely defined. A first definition dating back from the work of Karl Landsteiner a century ago presents Nabs as antibodies developing before known exposure to environmental antigens such as those present at birth¹⁰. A second definition emerged later based on the characteristic reactivity of Nabs towards generic self-determinants⁴. Although less accurate, this latter definition has supplanted the original designation and is now widely used. Yet, the two disparate definitions resulted in confusion and hindered the ability to compare Nabs between studies. In mice, Nabs are secreted primarily by B1 B cells¹¹. In humans, the source of Nabs is less clear. Aside from marginal zone B cells, peripheral blood naïve and memory B cells have been described to share typical reactivity patterns with Nabs^{12, 13, 14}.

A central characteristic of Nabs is their polyreactivity, i.e. the ability to react to apparently unrelated antigenic structures such as DNA, insulin, LPS, determinants on apoptotic cells or oxidation-specific epitopes (malondialdehyde)^{15, 16, 17, 18}. Polyreactivity is functionally advantageous as it increases the capacity to neutralize viruses¹⁹. For this reason, this important feature is positively selected among mutated B cells responding to viral infection. The molecular basis for polyreactivity, however, remains unsolved. The most common view posits that the antigen binding sites of Nabs are sufficiently flexible to allow the binding of different structures with low to intermediate affinity¹⁵.

### SUMMARY

According to certain embodiments, provided is a method for detecting the presence of natural antibodies reactive to one or more adducts in one or more subjects. The method involves analyzing a biological sample provided from said one or more subjects for the presence or absence of natural antibodies reactive to one or more adducts as set forth in Tables 1A and 1B (see below). In certain embodiments, the biological sample from the subject is selected from blood, tissue or fluid, preferably selected from hair, skin, nails, saliva, synovia, urine, liquor and blood. In select examples, the natural antibodies are IgG antibodies and/or IgM antibodies, typically from human or a non-human mammal. The analyzing step may be performed using any of a number of techniques known in the art, including, but not limited to, an immunoassay, preferably with direct or indirect coupling of one reactant to a detectable moiety. In specific embodiments, analyzing is carried out using an ELISA, RIA, multiplex immunoassay or immunofluorescence assay, western blot, line assay, dot blot assay.

The method may be performed using a solid support having a surface to which to at least one adduct as set forth in Tables 1A and Table 1B is bound. The method may further involve contacting a sample with the solid supports, washing solid supports, and subjecting the washed solid supports to one or more reagents that bind to a natural antibody and produces a detectable signal. In a specific example, the one or more reagents pertain an antibody that selectively binds to natural antibodies of a species to which the subject belongs. In a more specific embodiment, the antibody that selectively binds to natural antibodies of a species to which the subject belongs is conjugated or associated with a fluorometric or colorimetric marker (e.g. horseradish peroxidase). The methods may optionally further include comparing the detectable signal of the natural antibody with a control.

In an even more specific embodiment, the biological sample is cord blood from one or more neonates and peripheral blood from one or more subjects ranging from 6 mos to 70 years of age.

According to another embodiment, provided is a kit that includes a plurality of solid supports, wherein the plurality of solid supports are each individually coated with an adduct selected from adducts set forth in Tables 1A and 1B. The kit may include several different groups of solid supports that each include a different adduct.

A further embodiment is directed to a multiplexed assay method capable of measuring the interaction of one or more natural antibodies in a biological sample with one or more solid supports having solid phase surfaces. The method involves contacting the solid supports with the biological sample, the solid phase surfaces of the solid supports being treated to comprise individual chemical adducts set forth in Tables 1A and 1B, and determining the level of natural antibody captured in each of said wells relative to that captured to a well treated with a control adduct.

According to another embodiment, provided is a multiplexed assay method capable of measuring the interaction of one or more natural antibodies in a biological sample with one or more substrate surfaces that involves obtaining an adduct/microsphere reagent produced by attaching one or more adducts set forth in Tables 1A and 1B to a plurality of microspheres with a distinct fluorometric signature. The attaching may be direct or indirect. The biological sample is mixed with the adduct/microspheres to form a mixture and the mixture is washed to remove unbound biological sample from the microspheres. One or more natural antibodies from the biological sample that are captured by an adduct or multiple adducts can be detected using a fluorescently labeled reagent that binds to the one or more natural antibodies. In one example, the fluorescently labeled agent comprises Phycoerythrin.

According to a further embodiment, disclosed is a method for determining whether a subject has pancreatic cancer. The method involves analyzing a biological sample from the subject for presence of (i) anti-phosphorylated threonine antibodies, (ii) anti-phosphorylated tyrosine antibodies and (iii) anti-phosphorylated serine antibodies, and diagnosing a patient as having pancreatic cancer or risk of pancreatic cancer if the biological sample comprises (i), (ii) and (iii), or elevated levels of (i), (ii) or (iii) relative to a healthy control. The method may optionally further comprise administering a cancer therapy to the subject if diagnosed to have pancreatic cancer.

In yet a further embodiment, disclosed is method of identifying specificity of antibodies against a transplanted graft material in a subject. This method involves exposing a sample from the subject to one or more adducts as set forth in Tables 1A and 1B; and determining reactivity of antibodies in the sample to one or more of the adducts. In a specific example, the transplanted graft material comprises a transplanted organ. The method may further involve identifying an adduct to which the one or more antibodies reacts as a biomarker for an immune response against the transplanted graft material. In specific examples, the biomarker is selected from one or more of methylated proteins, phosphorylated proteins, or nucleotide derivatives. In more specific examples, the biomarker is selected from bilirubin, MDA-lysine, ADP-ribose, trimethylated-lysine and phosphorylated-serine. The method may further involve administering an effective amount of an immunosuppressive agent to the subject if reactivity is detected.

According to another embodiment, disclosed is a kit for use in a method for determining an immune response or level of immune response against transplanted graft material. The kit includes a solid support having a surface that is treated with a biomarker selected from methylated proteins, phosphorylated proteins, and/or several nucleotide derivatives, wherein, optionally, the solid support comprises a multiwell plate, a bead, or a microsphere, and wherein the kit optionally further comprises a detection reagent. In a more specific embodiment, the biomarker is selected from bilirubin, MDA-lysine, ADP-ribose, trimethylated-lysine or phosphorylated-serine, or a combination thereof.

### BRIEF DESCRIPTION OF DRAWINGS

FIG. 1. Monoclonal anti-adduct IgM display apparent polyreactive profiles. FIG. 1A. Reactivity of 7 IgM polyreactive mab cloned from healthy adult PB IgM+ memory B cells and a control non-reactive IgM mab to 81 adducts. M2.3 mab reacts preferentially to phosphorylated-serine,-threonine and -tyrosine as well as sulfonylated-cystein and tyrosine. FIG. 1B. Reactivity of 3 representative IgM polyreactive mab cloned from healthy adult PB IgM+ memory B cells and a control non-reactive IgM mab to 8027 recombinant proteins included in the V4.0 protoarray. Signal to Background ratio of 5 and difference of 100 were used as cutoff values.

FIG. 2. Serological profiling of anti-adduct IgM and IgG across the lifespan. FIG. 2A. Heat map representation of IgM and IgG reactivity to 81 natural adducts in cord blood (CB, N=12) and peripheral blood from healthy subjects aged 1 to 68 years (PB, N=30). Each column corresponds to a unique subject. FIG. 2B. IgM reactivity to specific adducts across the lifespan.

FIG. 3. Serological profiling of anti-adduct IgM and IgG across the lifespan. FIG. 3A. Correlation matrix for the comparison of IgM and IgG reactivity to adducts shown in FIG. 2A between CB and PB specimens (age 1 to 68 years). FIG. 3B. Pearson correlation coefficients for all pairwise comparisons between CB and PB for IgM and IgG.

FIG. 4 Diversification of anti-adduct IgM repertoire during infancy. FIG. 4A. Pearson correlation coefficient heat map comparing CB and infant PB IgM reactivity to adducts. FIG. 4B. Heat map representation of normalized IgM reactivity to 81 adducts in infant PB (N=18). Adducts ae clustered (unsupervised) according to the age when increase in reactivity was detected. FIG. 4C. Development of IgM reactivity to adducts included in the top two clusters (blue and grey).

FIG. 4D. Top ten most informative adducts discriminating between serum IgM reactivity before and after 5 months of age. The adducts were identified through random forest classification, using the Boruta algorithm.

FIG. 5. Differential levels of immunogenic adducts between cord blood and adult peripheral blood. FIG. 5A. Quantification of 7 specific adducts in PBMC lysates and EVs prepared from CB or healthy aPB. **** p<0.0001, ** p<0.01. FIG. 5B. Summary of adduct quantification. Higher adduct levels in a PB compared to CB are depicted with red dots. However, adduct levels in a PB compared to CB are depicted with green dots. Unchanged levels are depicted with black dots. The dot sizes are proportional to the normalized values.

FIG. 6. Heatmap representation of normalized IgG reactivity to 93 adducts in Pancreatic Cancer patients (n=39) and Healthy Donors (n=40).

FIG. 7A. Boruta algorithm identified a set of 19 target adducts recognized by IgG that most efficiently discriminated between healthy donors and pancreatic cancer cases. FIG. 7B. IgG reactivity to the 3 phosphorylated residues as the most significant target adduct discriminating between pancreatic cancer patients (red) and healthy controls (blue)}. FIG. 7C. Differences in IgG reactivity between patients with (n=17-purple) or without metastasis (n=22-green). *** p-value<0.001; ** p-value<0.05 (Welch's test). FIG. 7D. Uniform Manifold Approximation and Projection (UMAP) comparing reactivity to the 3 phosphorylated residues between pancreatic cancer patients (red) and healthy controls (blue).

FIG. 8. Detection of phosphorylated-tyrosine in tumor and adjacent normal tissue by immunofluorescence. 13 tumor/normal paired specimens were tested. 11 cases tested positive for phospho-Tyr in the tumor area and negative in the non-tumoral area. 2 cases were positive in both areas.

FIG. 9 Reactivity to generic autoantigens and apoptotic cells. FIG. 9A. Reactivity of monoclonal IgM Nabs secreted by immortalized memory B cells derived from healthy adult donor PBMC to insulin, MDA-BSA, LPS and dsDNA assessed by ELISA. FIG. 9B. Anti-MDA and anti-phosphoryl antibody reactivity to insulin, LPS and dsDNA assessed by ELISA. FIG. 9C. monoclonal IgM Nabs reactivity to apoptotic cells assessed by flow cytometry.

FIG. 10A. Reactivity of an IgG mab cloned from healthy adult plasma cell to 81 adducts. The mab reacts primarily to phosphorylated-threonine. FIG. 10B. Competitive inhibition of the IgG mab reactivity to LPS (gre bars) and insulin (red bars) with phosphorylated-threonine peptide.

FIG. 11. Unsupervised clustering of IgM reactivity to 81 adducts across the lifespan. Heat map representation of IgM reactivity to 81 adducts in CB (N=12) and PB from healthy subjects aged 1 to 68 years (N=30). Each column corresponds to a subject. Each row corresponds to a single adduct. Rows are clustered using the Ward's minimum variance method.

FIG. 12. Assay reproducibility, comparison between IgM and IgG reactivity. FIG. 12A, Duplicate ELISA measurements of reactivity to all 81 adducts for IgM and IgG in control pooled sera from healthy adult donors. FIG. 12B. Correlation between reactivity observed for IgM and IgG for a representative healthy adult donor. FIG. 12C. Principal component analysis projection of CB IgM reactivity (red, N=12), PB IgM reactivity (black N=30), CB IgG reactivity (blue, N=12) and PB IgG reactivity (grey N=30).

FIG. 13. Anti-adduct IgM and IgG reactivity in females and males. Principal component analysis projection of PB IgM (right) and IgG (left) reactivity to 81 adducts for females (pink, N=18), males (blue, N=12) and CB irrespective of sex (black N=12).

FIG. 14. Anti-adduct IgM reactivity in neonates and infants. Principal component analysis projection of IgM reactivity for CB (black N=12) and PB collected from birth to 5 months of age (red, N=12) and from 5 months to 1 year of age (green, N=6). PCA was performed using all 81 adducts (FIG. 14A) or only the 10 most informative adducts identified through a random forest classifier (FIG. 14B).

FIG. 15. Development of IgM reactivity to natural adducts. IgM reactivity to adducts included in clusters 3 to 6 following unsupervised clustering using the Ward's minimum variance method (FIG. 2) are reported as z-scores. The median trajectory for each cluster is depicted with a solid brown line.

FIG. 16. CDR3 of the adduct-specific clones.

FIG. 17. Reactivity of antibodies generated from B cells infiltrating cardiac grafts.

### DETAILED DESCRIPTION

Embodiments of the invention are based on the discovery that some Nabs displaying an apparent polyreactive profile are in fact specific to chemical moieties exposed on multiple molecules, such as post-translational modifications (PTM) or other adducts. Recognition of these motifs on unrelated antigens results in a polyreactive pattern. To test this hypothesis, a high-dimensional platform was developed to assess the reactivity to 93 common adducts shared by numerous proteins and other macromolecules. Using this assay, the anti-adduct reactivity profile of representative polyreactive monoclonal IgM derived from human peripheral blood memory B cells was investigated. The development and variations of IgG and IgM to adducts from birth to old age was also investigated.

### DEFINITIONS

The term “antibody” herein is used in the broadest sense and specifically covers natural antibodies (Nabs), but may also refer to monoclonal antibodies (including full length monoclonal antibodies), polyclonal antibodies, multi-specific antibodies (e.g., bispecific antibodies), and antibody fragments so long as they exhibit the desired biological activity, depending on the context in which they are referred. In preferred embodiments, antibody refers natural antibodies. As used herein, the term “detecting” refers to observing a signal from a label moiety to indicate the presence of a biomarker in the sample. Any method known in the art for detecting a particular detectable moiety can be used for detection. Exemplary detection methods include, but are not limited to, spectroscopic, photochemical, biochemical, immunochemical, electrical, optical or chemical methods.

A “label,” “imaging agent” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA, e.g. horseradish peroxidase), biotin, digoxigenin, or haptens and proteins which can be made detectable, e.g., by incorporating a radiolabel into the peptide or used to detect antibodies specifically reactive with the peptide.

The term “sample” as used herein includes any biological specimen obtained from a patient. Samples include, without limitation, whole blood, plasma, serum, red blood cells, white blood cells (e.g., peripheral blood mononuclear cells), cord blood, ductal lavage fluid, nipple aspirate, lymph (e.g., disseminated tumor cells of the lymph node), bone marrow aspirate, saliva, urine, stool (i.e., feces), sputum, bronchial lavage fluid, tears, fine needle aspirate (e.g., harvested by fine needle aspiration that is directed to a target, such as a tumor, or is random sampling of normal cells, such as periareolar), any other bodily fluid, a tissue (e.g., tumor tissue) such as a biopsy of a tumor (e.g., needle biopsy) or a lymph node (e.g., sentinel lymph node biopsy), and cellular extracts thereof. In some embodiments, the sample is whole blood or a fractional component thereof such as plasma, serum, or a cell pellet.

As used herein, the term “subject” refers to a human or non-human mammal or animal. Non-human mammals include livestock animals, companion animals, laboratory animals, and non-human primates. Non-human subjects also specifically include, without limitation, chickens, horses, cows, pigs, goats, dogs, cats, guinea pigs, hamsters, mink, and rabbits. In some embodiments, a subject is a patient. As used herein, a “patient” refers to a subject who is under the care of a physician or other health care worker, including someone who has consulted with, received advice from or received a prescription or other recommendation from a physician or other health care worker.

The term “transplant” Is known to the person skilled in the art. In the context of the present invention, the term transplant relates to any cell, tissue, or organ being transferred (transplanted or “grafted”) from the body of one subject into the body of another subject (allograft) or from a subject and back into a subject (autograft).

As used herein, term “immune response” relates to a selective response mounted by the immune system of a subject against a transplant, a self-antigen, a pathogen, or an allergen. During an immune response specific antibodies and/or cytotoxic T cells are produced. The term immune response includes T cell mediated and/or B cell mediated immune responses that are influenced by modulation of T cell costimulation. Exemplary immune responses include T cell responses, e.g., proliferation, cytokine production, and cellular cytotoxicity. In addition, the term “immune response” includes immune responses that are indirectly effected by T cell activation, e.g., antibody production (humoral responses) and activation of cytokine responsive cells, e.g., macrophages.

“Transplant/graft rejection” as used herein means the rejection of transplanted organs or tissue by the recipient's immune system, which destroys the transplanted tissue.

As used herein, the term “isolating” or other grammatical forms thereof, refers to removing molecules or structures of interest from their natural environment or from a starting material (i.e., they are isolated) and where they are largely free from other components with which they are naturally associated or substantially free of other components that may render future use or study sub-optimal, difficult or impossible.

“Immunosuppressive agent,” “immunosuppressive drug,” and “drug,” are used interchangeably herein, and refer to any agent which inhibits or prevents an immune response against the transplanted tissue following a transplant procedure. Exemplary agents include, but are not limited to, dexamethasone, cyclosporin A, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, trimetrexate), purine analogs (e.g., cladribine, fludarabine, 6-mercaptopurine, thiamiprine, thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, tegafur), fluocinolone, triaminolone, anecortave acetate, flurometholone, medrysone, and prednisolone.

As used herein, the terms “treat,” “treatment,” “treating,” or “amelioration” refer to therapeutic treatments for transplant rejection known in the art, wherein the object is to reverse, alleviate, ameliorate, inhibit, slow down or stop the progression of transplant rejection, or reduce the severity of a symptom or condition associated with transplant rejection. The term “treating” includes reducing or alleviating at least one adverse effect or symptom of a condition. Treatment is generally “effective” if one or more symptoms or clinical markers of transplant rejection are reduced.

### Description of Embodiments

According to certain embodiments, disclosed is a method for detecting the presence of natural antibodies reactive to one or more adducts in one or more subjects. The method involves analyzing a biological sample provided from said one or more subjects for the presence or absence of natural antibodies reactive to one or more adducts as set forth in Tables 1A and 1B. The biological sample from the subject is typically selected from peripheral blood or cord blood. In a specific embodiment, the biological sample of the one or more subjects comprises cord blood from one or more neonates and peripheral blood from one or more subjects ranging from 6 mos to 70 years of age.

The natural antibodies detected are typically IgG antibodies and/or IgM antibodies. Analyzing typically involves performing an immunoassay, preferably with direct or indirect coupling of one reactant to a detectable moiety. In certain embodiments, analyzing involves using an ELISA, RIA, multiplex immunoassay or immunofluorescence assay, western blot, line assay, dot blot assay.

In a specific embodiment, analyzing comprises subjecting the biological sample to a plurality of wells each having an adduct as set forth in Tables 1A and 1B bound to a surface of the well. The method may further involve washing the wells after exposure to a sample, and subjecting the washed wells to one or more reagents that bind to a natural antibody and produces a detectable signal. The one or more reagents typically comprise an adduct or alternatively an antibody that selectively binds to natural antibodies of a species to which the subject belongs. In a more specific embodiment, the adduct to which a natural antibody selectively binds or an antibody specific to such natural antibody is conjugated or associated with a colorimetric marker, such as horseradish peroxidase. The method may further involve comparing the detectable signal of the natural antibody with that of a control. A control can be control adduct (e.g MDA-lysine) against which IgM reactivity does not change significantly from birth to adulthood.

Other embodiments pertain to a kit comprising a multiwell plate wherein a plurality of wells of the multiwell plate are each individually coated with a different adduct selected from adducts set forth in Tables 1A and 1B (below).

TABLE 1A

Monomethylated-Lys
Dimethylated-Lys
Trimethylated-Lys
Formylated-Lys
Propionylated-Lys
Butyrylated-Lys
2-Hydroxyisobutyrylated-Lys
Crotonylated-Lys
Malonylated-Lys
Succinylated-Lys
Gutarylated-Lys
Beta-hydroxybutrylated-Lys
Biotinylated-Lys
Carbamylated-Lys
N-homocysteinylated-Lys
Acetylated-Lys
MDA-Lys
Pyrraline
CEL
CML
Fructose lysine
NEDD8
SUMO2
Protoporphyrin IX
Billirubin
PLP
Monomethylated-Arg
Asymmetric demethylated-Arg
Symmetric demethylated-Arg
Citrullinated-Arg
MDA-Arg
Pyruvoylated-Ser
Phosphorylated-Ser
GlcNAcylated-Ser
GalNacylated-Ser
Phosphatidylserine
Phosphorylated-Thr
Methylated-His
Sulfenyl-Met (SO)
Sulfinyl-Met (SO₂)
Demthylated-Met (Homocysteine)
Nitrated-Tyr
Chlorinated-Tyr
Hydroxylated-Tyr (L-DOPA)
Phophorylated-Tyr
Iodinated-Tyr
Sulfonylated-Tyr (SO3)
FMN
FAD
Cysteine
Homocysteinlyated-Cys
S-palmoitoylated-Cys
Sulfonylated-Cys (SO3H)
Succinated-Cys
N-myristoylated-Gly
Dioxygenated-Trp
Gamma-carboxylated-Glu
ADP ribose
Ubiquitin
SUMO1
Neu5Gc
Neu5Ac
Ribose
PQQ
V B9
TPP
VB12
NAD
ATP
CTP
SAM
V B1
V C
Tetrahydrobiopterin
Rentinal
V K2
CoQ10
Co M
VB6
4-HNE GSH

TABLE 1B

| | | | | | Coating |
|---|---|---|---|---|---|
| Adduct | Formula | M.W. | Purity | Source | concentration |
| 4-HNE cysteine (4-hydroxy | C14H25NO5S | 319.4 | ≥98% | Cayman | 10 mM - In |
| Nonenal Mercapturic Acid) | | | | Chemical | ethanol |
| M1G (pyrimido[1,2-a]purin- | C₈H₅N₅O | 187.76 | . | Toronto | 10 mM - In |
| 10(3H)-one) | | | | Research | DMSO |
| | | | | Chemicals | |
| Pentosidine | C17H26N6O4 | 378.433 | ≥98% | Cayman | 10 mM - In |
| | | | | Chemical | DMSO |
| UDP-GlcNAc (UDP-N-acetyl- | C17H25N3O17P2•2Na | 651.3 | ≥95% | Cayman | 10 mM - In |
| D-Glucosamine) | [XH2O] | | | Chemical | H20 |
| Co-Methyl (Methylcobalamin) | C₆₃H₉₁CoN₁₃O₁₄P | 1344.4 | . | Sigma | 10 mM - In |
| | | | | | H20 |
| Co-A (Coenzyme A) | C21H36N7O16P3S | 767.535 | ≥90% | Cayman | 10 mM - In |
| | | | | Chemical | H20 |
| ADP (Adenosine diphosphate) | C₁₀H₁₅N₅O₁₀P₂ | 427.2 | ≥95% | Sigma | 10 mM - In |
| | | | | | H20 |
| Ox GSH (Oxidized glutathione) | C₂₀H₃₂N₆O₁₂S₂ | 612.63 | ≥98% | Sigma | 10 mM - In |
| | | | | | H20 |
| D-ribose 5-phosphate | C₅H₉Na₂O₈P•2H₂O | 310.1 | ≥99% | Sigma | 10 mM - In |
| | | | | | H20 |
| AMP (Adenosine | C₁₀H₁₄N₅O₇P•H₂O | 365.24 | ≥97% | Sigma | 10 mM - In |
| monophosphate) | | | | | coating buffer |
| V K1 (Phytonadione) | C₃₁H₄₆O₂ | 450.7 | . | Sigma | 10 mM - In |
| | | | | | DMSO |
| V K3 (Menadione) | C₁₁H₈O₂ | 172.2 | . | Sigma | 10 mM - In |
| | | | | | DMSO |

### Transplant Immunoreactivity

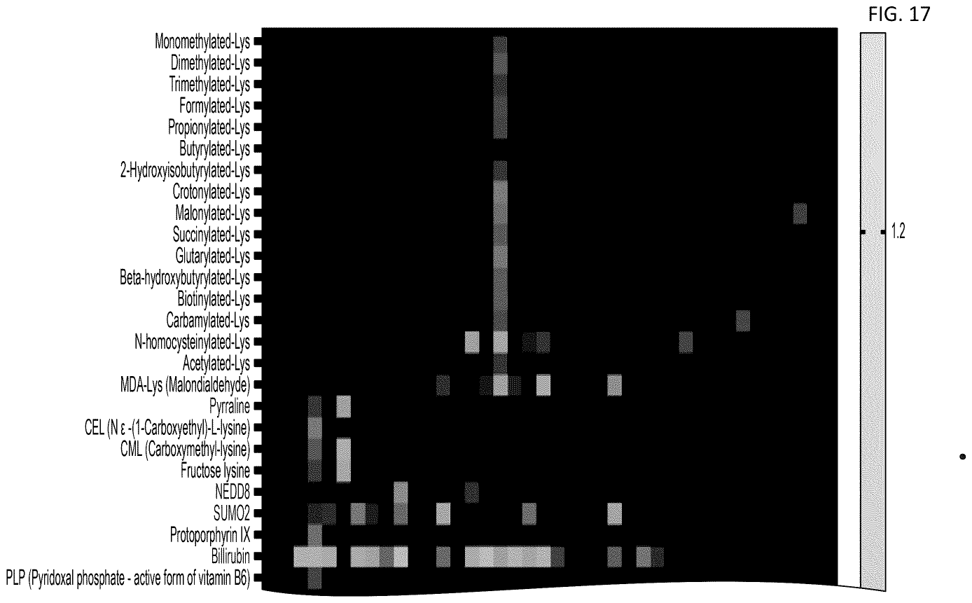

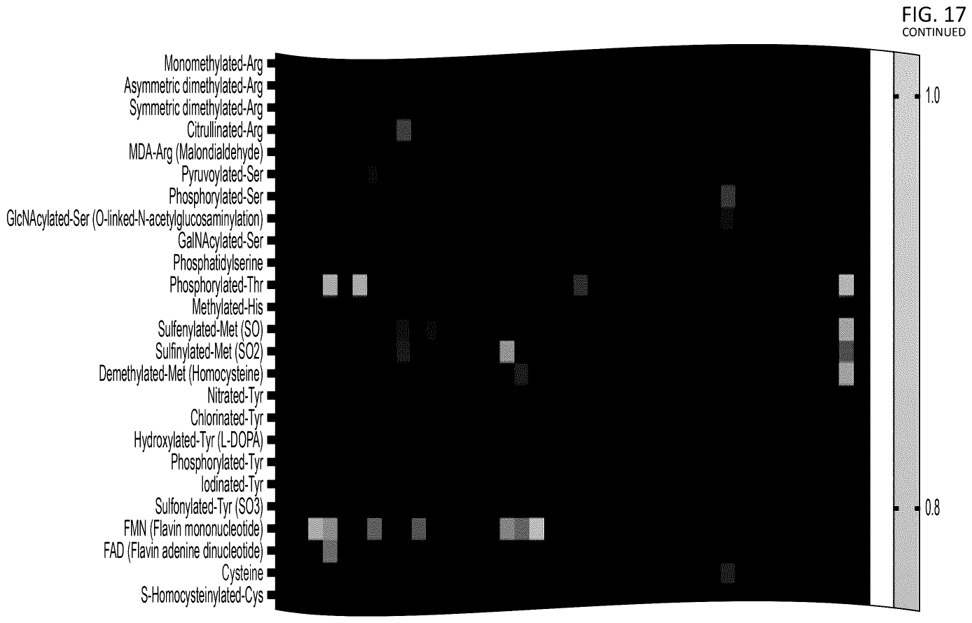

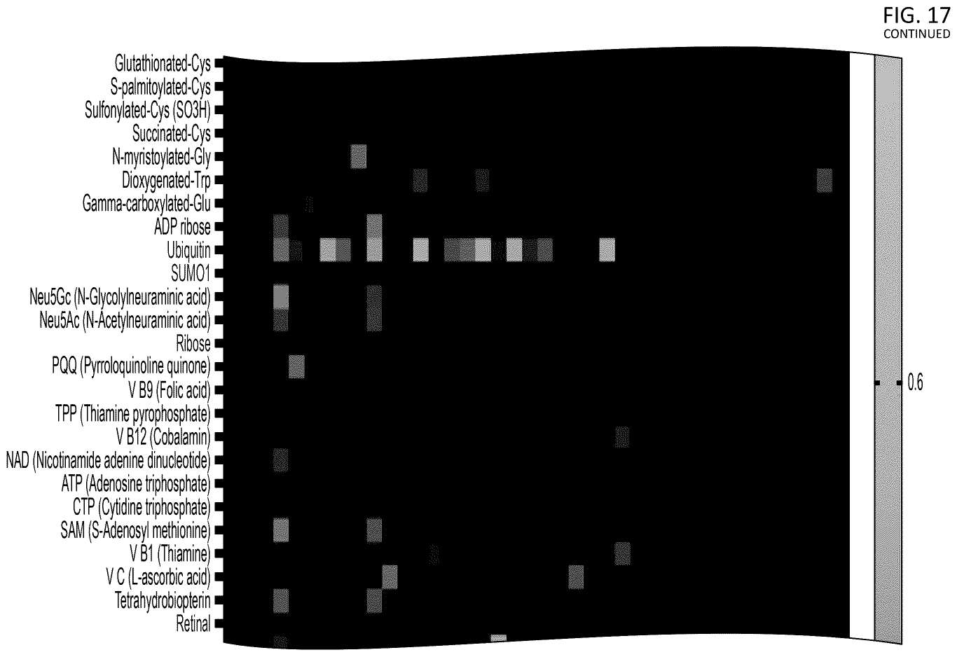

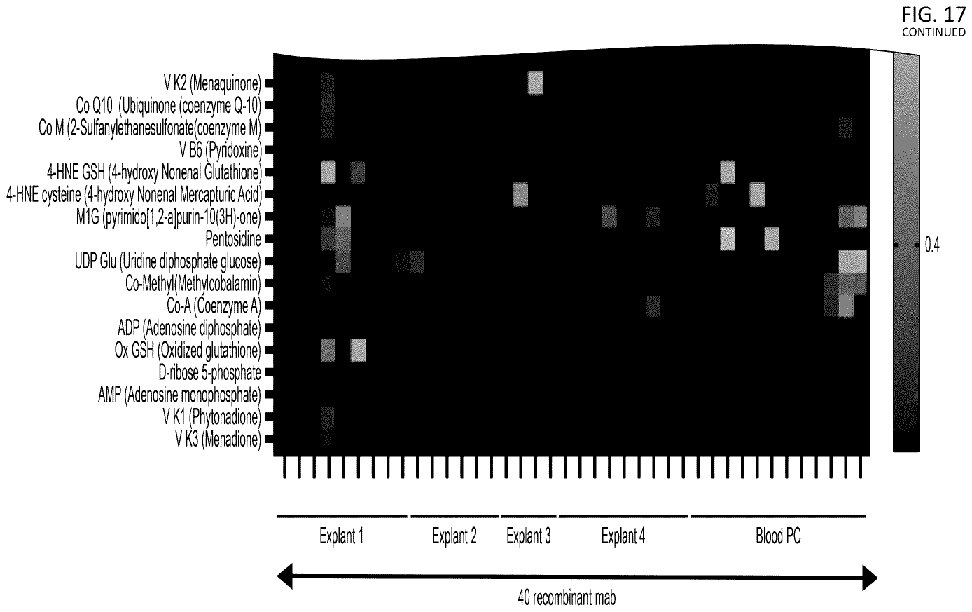

Based on the data presented in FIG. 17 and described in Example 4, methods of identifying specificity of antibodies developed in response to a transplanted graft material in a subject are provided. The method involves exposing a sample to one or more adducts as set forth in Tables 1A and 1B; and determining reactivity of the sample to one or more of the adducts. In one specific example, the sample contains antibodies. In a specific example, the transplanted graft material is a transplanted organ. Transplanted organs may include but are not limited to a heart, a lung, a liver or a kidney. The method further involves identifying an adduct to which the one or more antibodies reacts as a biomarker for an immune response against the transplanted graft material.

According to another embodiment, provided is a method of determining an immune response or level of immune response against transplanted graft material in a subject. The method involves detecting reactivity against the biomarker demonstrated to have reactivity with one or more antibodies in a sample from the subject. In a specific example, the biomarker is one or more of methylated proteins, phosphorylated proteins, and/or several nucleotide derivatives, including but not limited to, bilirubin, MDA-lysine, ADP-ribose, trimethylated-lysine and phosphorylated-serine. In another specific example, the transplanted graft material is a heart. In a further embodiment, if reactivity against the biomarker is demonstrated, an effective amount of an immunosuppressive agent is administered to the subject.

### Detection Kits

The present invention also provides kits comprising the components of the combinations of the invention in kit form. A kit of the present invention includes one or more components including, but not limited to, one or more adducts as set forth in Tables 1A and 1B.

Also provided are diagnostic or detection reagents and kits comprising one or more such reagents for use in a variety of detection assays. The kit's components, such as one or more adducts set forth in Tables 1A and 1B may be pre-attached to a solid support, or may be applied to the surface of a solid support when the kit is used. In some embodiments, the signal generating means may come pre-associated with an antibody of the invention or may require combination with one or more components, e.g., buffers, antibody-enzyme conjugates, enzyme substrates, or the like, prior to use. Kits may also include additional reagents, e.g., blocking reagents for reducing nonspecific binding to the solid phase surface, washing reagents, enzyme substrates, and the like. The solid phase surface may be in the form of a tube, a bead, a microtiter plate, a microsphere, or other materials suitable for immobilizing proteins, peptides, or polypeptides. In particular aspects, an enzyme that catalyzes the formation of a chemiluminescent or chromogenic product or the reduction of a chemiluminescent or chromogenic substrate is a component of the signal generating means. Such enzymes are well known in the art. Optionally the kit may also comprise instructions for carrying out the methods of the invention.

Also provided is a kit comprising an adduct capable of binding a natural anti-adduct antibody packaged in a container, such as a vial or bottle, and further comprising a label attached to or packaged with the container, the label describing the contents of the container. The detection kits disclosed herein may also be prepared that comprise at least one of the adducts disclosed herein and instructions for using the composition as a detection reagent. Containers for use in such kits may typically comprise at least one vial, test tube, flask, bottle, syringe or other suitable container, into which one or more of the detection and/or therapeutic composition(s) may be placed, and preferably suitably aliquoted. Where a second detection agent is also provided, the kit may also contain a second distinct container into which this second detection composition may be placed. Alternatively, a plurality of compounds may be prepared in a single composition, and may be packaged in a single container means, such as a vial, flask, syringe, bottle, or other suitable single container. The kits of the present invention will also typically include a means for containing the vial(s) in close confinement for commercial sale, such as, e.g., injection or blow-molded plastic containers into which the desired vial(s) are retained. Where a radiolabel, chromogenic, fluorigenic, or other type of detectable label or detecting means is included within the kit, the labeling agent may be provided either in the same container as the detection or therapeutic composition itself, or may alternatively be placed in a second distinct container means into which this second composition may be placed and suitably aliquoted. Alternatively, the detection reagent and the label may be prepared in a single container means, and in most cases, the kit will also typically include a means for containing the vial(s) in close confinement for commercial sale and or convenient packaging and delivery.

A device or apparatus for carrying out the detection or monitoring methods described herein is also provided. Such an apparatus may include a chamber or tube into which sample can be input, a fluid handling system optionally including valves or pumps to direct flow of the sample through the device, optionally filters to separate plasma or serum from blood, mixing chambers for the addition of capture agents or detection reagents, and optionally a detection device for detecting the amount of detectable label bound to the capture agent immunocomplex. The flow of sample may be passive (e.g., by capillary, hydrostatic, or other forces that do not require further manipulation of the device once sample is applied) or active (e.g., by application of force generated via mechanical pumps, electroosmotic pumps, centrifugal force, or increased air pressure), or by a combination of active and passive forces.

In a related embodiment, provided is a kit for use in a method for determining an immune response or level of immune response against transplanted graft material. The kit includes a solid support having a surface that is treated with individual biomarkers selected from methylated proteins, phosphorylated proteins, and/or several nucleotide derivatives. In a specific example, the solid support is a well surface of a multiwell substrate. In another example the substrate is a bead or microsphere. In a more specific embodiment, the biomarkers used for treating the substrate surface are selected from bilirubin, MDA-lysine, ADP-ribose, trimethylated-lysine and phosphorylated-serine. The kit may optionally include a detection reagent.

According to another embodiment, provided is a kit for use in a method of determining whether a subject has pancreatic cancer. The kit includes a solid support having a surface that is treated with individual biomarkers selected from phosphorylated threonine, phosphorylated tyrosine antibodies and phosphorylated serine or combination thereof. In a specific example, the solid support is a well surface of a multiwell substrate. In another example the substrate is a bead or microsphere. The kit may optionally comprise a detection agent.

In related embodiments, also provided is a processor, a computer readable memory, and a routine stored on the computer readable memory and adapted to be executed on the processor to perform any of the methods described herein. Examples of suitable computing systems, environments, and/or configurations include personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, or any other systems known in the art.

### EXAMPLES

### Example 1

### Monoclonal Polyreactive IgM Nabs Recognize Specific Adducts.

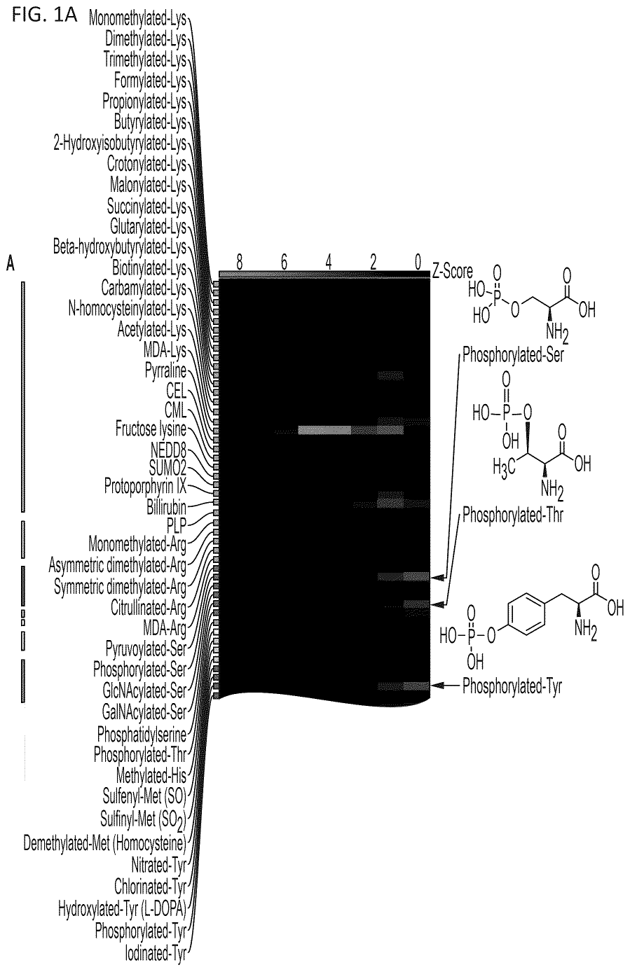

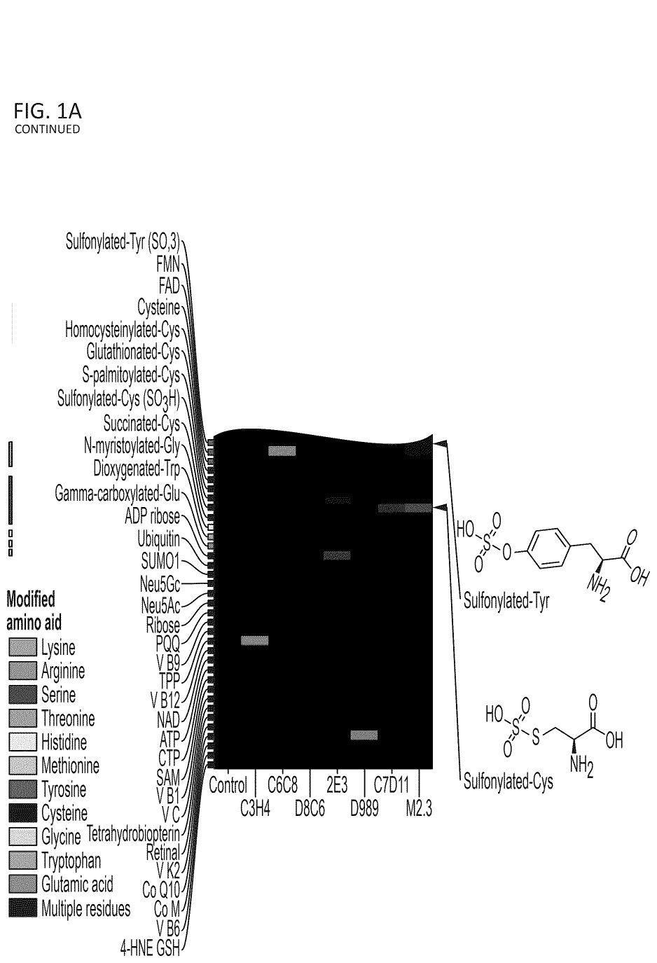

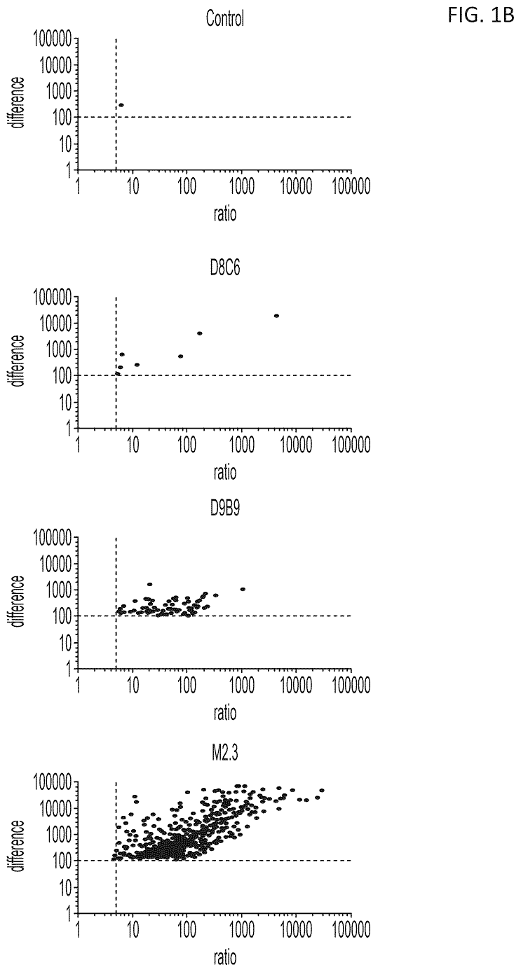

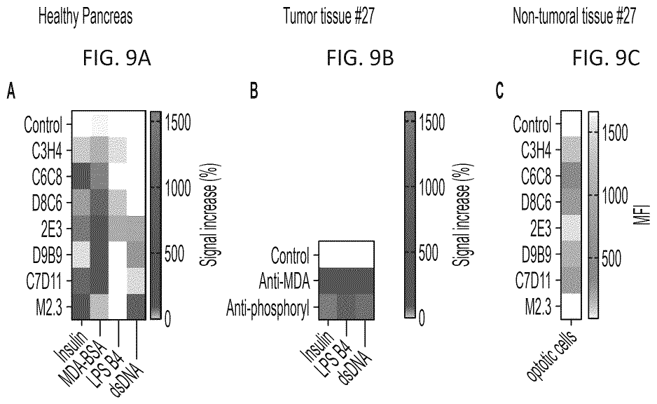

We designed an ELISA platform to assess antibody reactivity to 81 natural adducts (Table 2). Our panel includes 60 distinct PTM on 11 amino acids and 21 co-factors. The latter covalently or non-covalently bind to proteins or macromolecules and behave essentially as biochemical adducts. While this panel does not cover all possible natural adducts, it nevertheless includes most common PTM and offers a representative sample of the entire “adductome”. We used this high-dimensional ELISA platform to interrogate the reactivity of 7 representative monoclonal polyreactive IgM produced by immortalized B cell clones established from peripheral blood IgM+CD27+CD19+ memory B cells of healthy adult donors. These monoclonal antibodies (mab) were selected for their combined reactivity to insulin, malondialdehyde (MDA) and apoptotic cells, a pattern typically attributed to Nabs (FIG. 9). As depicted in FIG. 1A, all 7 mab reacted to at least one adduct, while displaying distinct reactivity profiles. Clone C6C8, for instance, solely reacted to flavin mononucleotide (FMN), an adduct of flavoproteins. Clone M2.3 on the other hand, reacted mostly to phosphoryl-serine, -threonine, -tyrosine as well as sulfonyl-cysteine and -tyrosine. These five PTM shared a core composed of 3 atoms of oxygen (FIG. 1A), suggesting that M2.3 is specific to this motif. The recognition of adducts exposed on multiple proteins and other biomolecules such as phosphoryl or sulfonyl groups can provide a suitable explanation for the clones' apparent polyreactivity. We further confirmed by ELISA that MDA and phosphoryl groups recognized by several of the IgM mab are exposed on insulin, LPS and DNA, the three most common autoantigens used to assess polyreactivity (FIG. 9B)> As illustrated in FIG. 1B, 3 representative mab specific to distinct adducts reacted to numerous recombinant human proteins spotted on a microarray. Clone M2.3 alone reacted to 9.98% (801/8027) of all recombinant proteins included in the array, demonstrating the polyreactive pattern of antibodies recognizing widely distributed adducts.

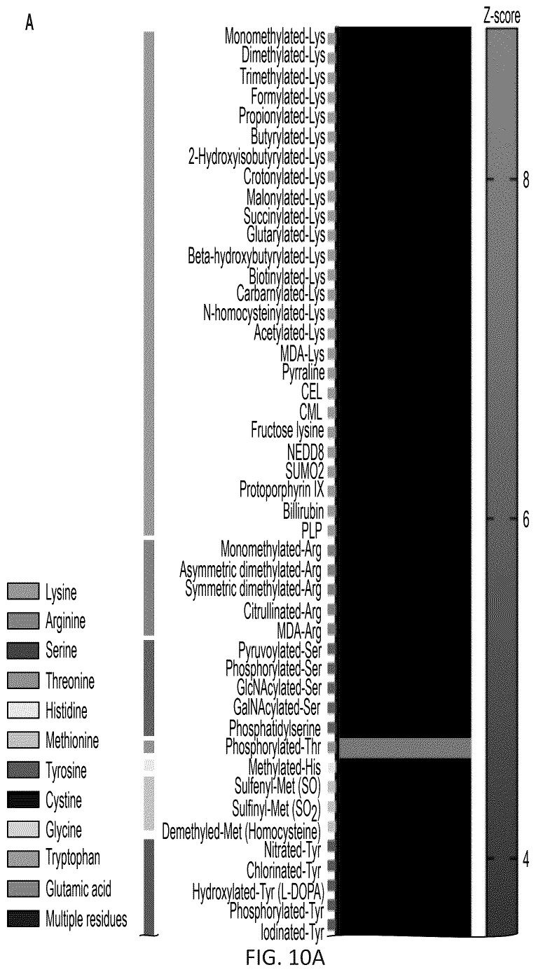

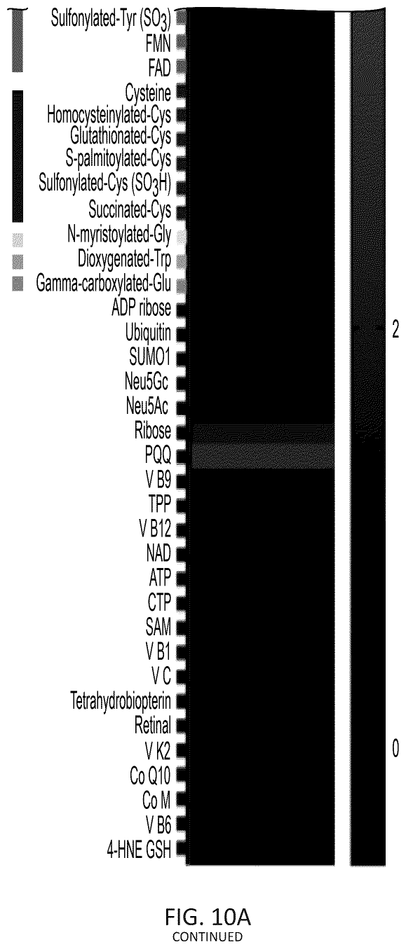

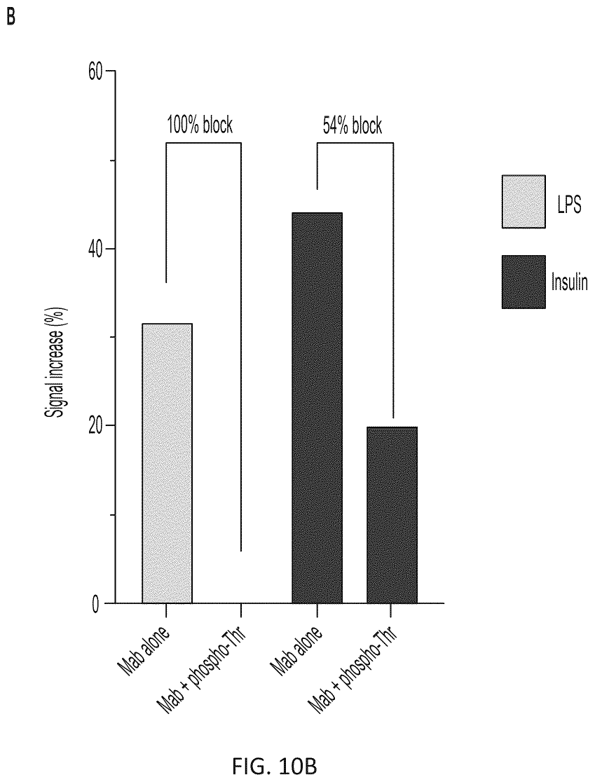

To confirm the mab specificity to adducts, a series of competitive ELISA were conducted where the binding of the mab would be displaced by the corresponding adduct in solution. However, these experiments were inconclusive due mainly to the high avidity of decavalent IgM clones and resulting overall strength of the antigen-antibody complex. To circumvent this issue, a recombinant monoclonal IgG generated from healthy donor blood plasma cells with a reactivity profile comparable to M2.3 was used. While M2.3 IgM reacted to phosphoryl groups affixed to serine, tyrosine or threonine indiscriminately, the IgG mab only reacted to phosphoryl-threonine (FIG. 10A). This IgG mab also reacted to insulin and LPS, hence displaying a typical polyreactive profile. Because the avidity of the IgG mab was much lower than that of IgM, confirmation was obtained that reactivity to insulin and LPS (e.g. polyreactivity) could be blocked by phosphoryl-threonine in solution (FIG. 10B).

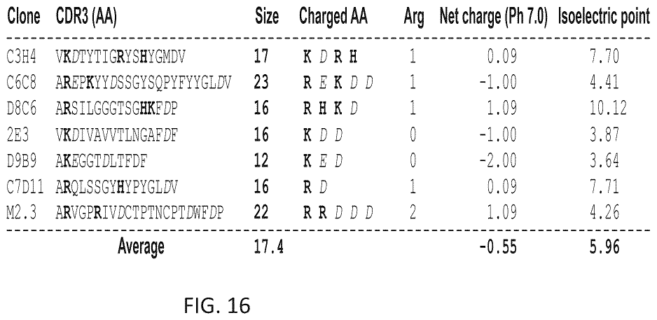

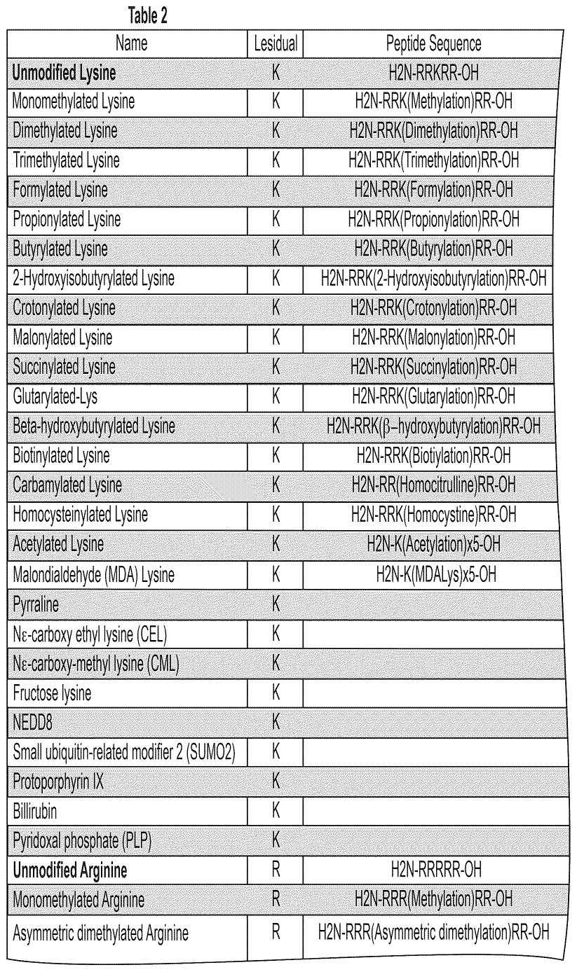

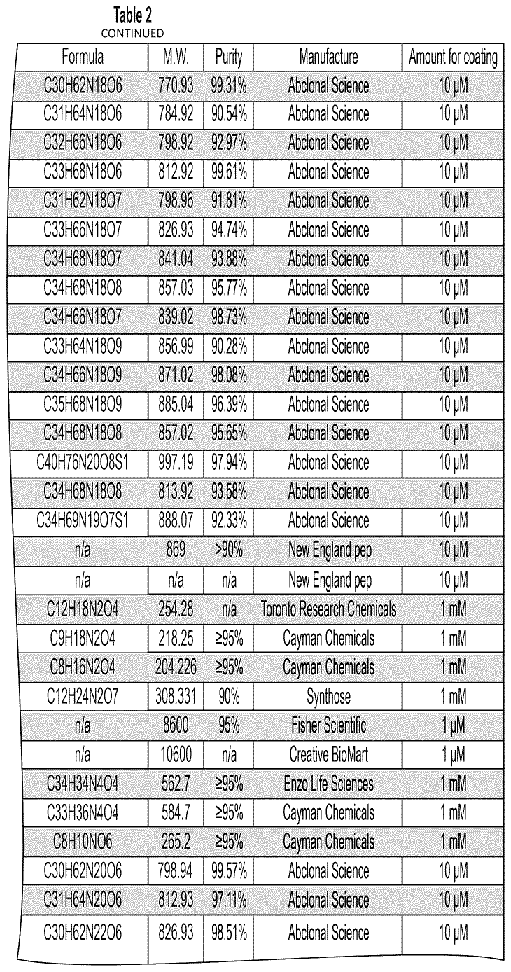

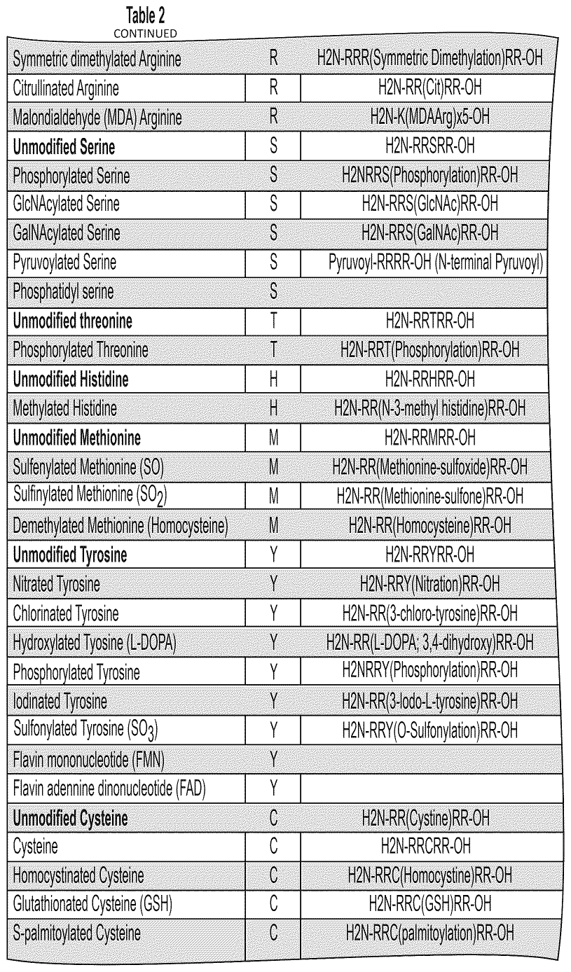

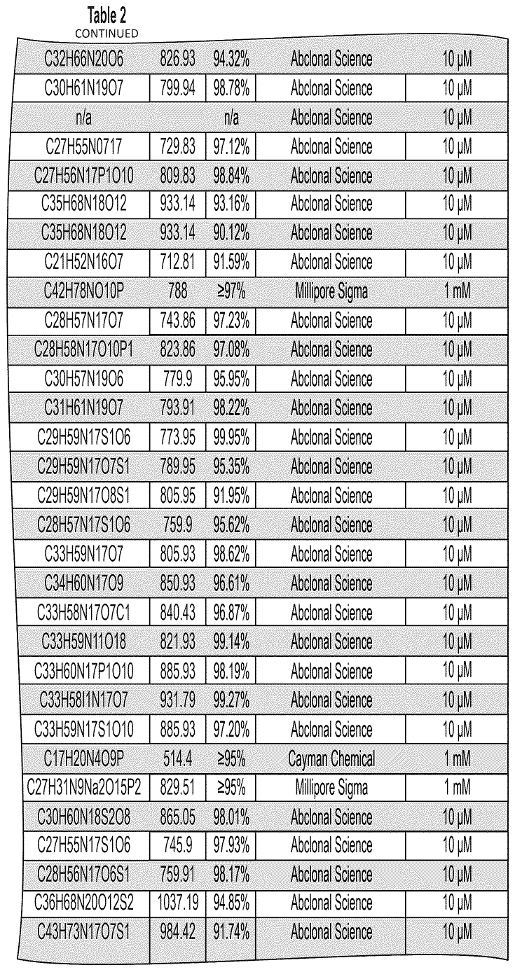

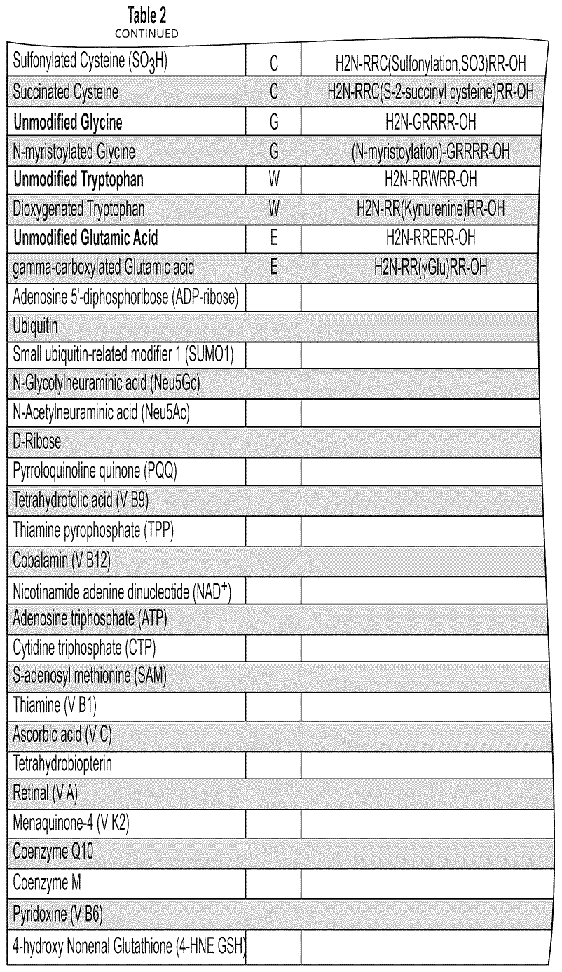

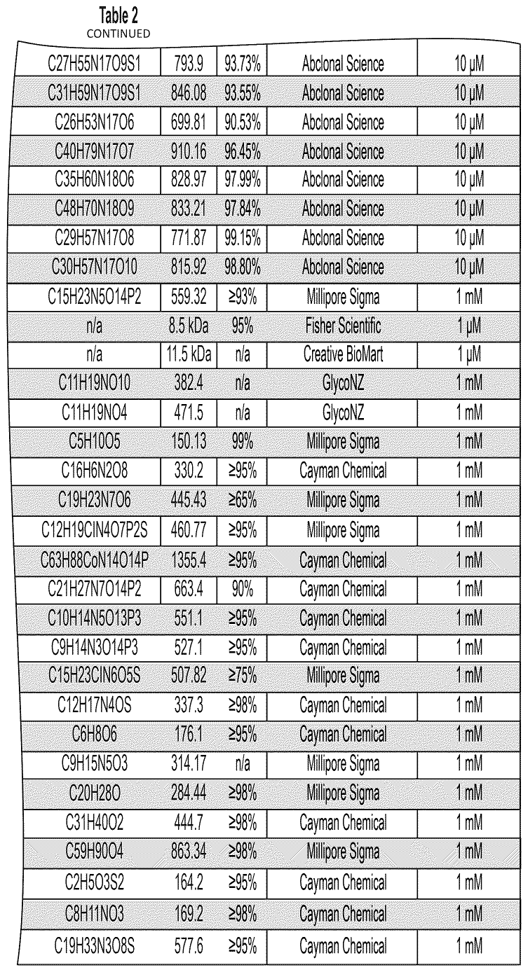

Previous studies have reported that the immunoglobulin heavy chain CDR3 sequence of polyreactive antibodies may differ from that of non-polyreactive antibodies (19-21). The CDR3 of the adduct-specific clones included in the study were analyzed and compared to available data collected from polyreactive and non-polyreactive antibodies (FIG. 16). Although our sample was small, several observations were made: 1) the average sequence length in AA was 17.4. This falls within the range of both polyreactive and non-polyreactive antibodies reported by Guthmiller et al. (19); 2) all monoclonal antibodies had charge basic residues in their CDR3, including 1 or 2 Arginine for 5 of them; and 3) these basic amino acids explain their relatively high isoelectric point (average of 5.96), which appears to be consistent with what was reported by Guthmiller for polyreactive antibodies.

### Marked Differences Between IgM and IgG Anti-Adduct Reactivity Profiles Across the Lifespan

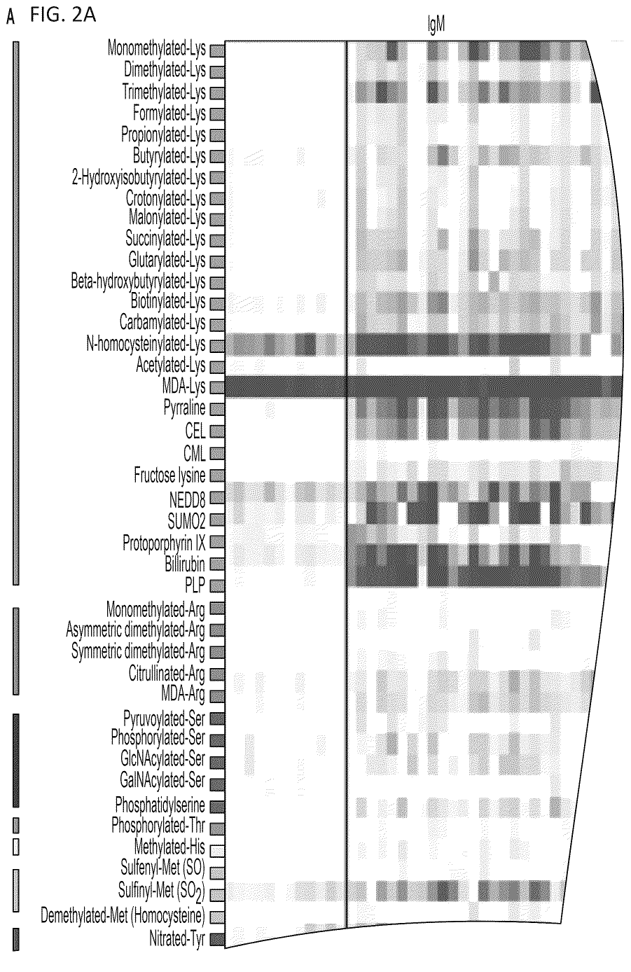

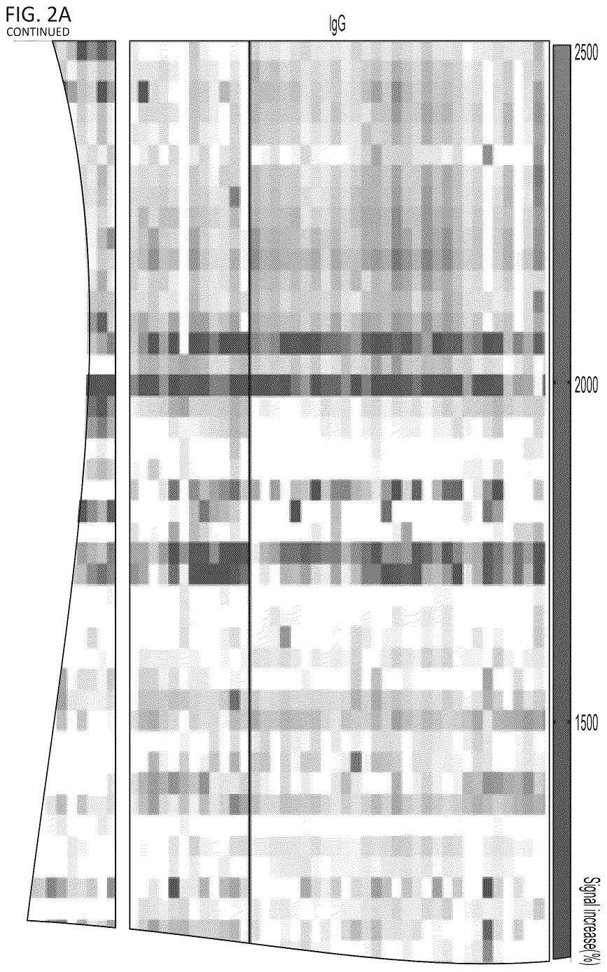

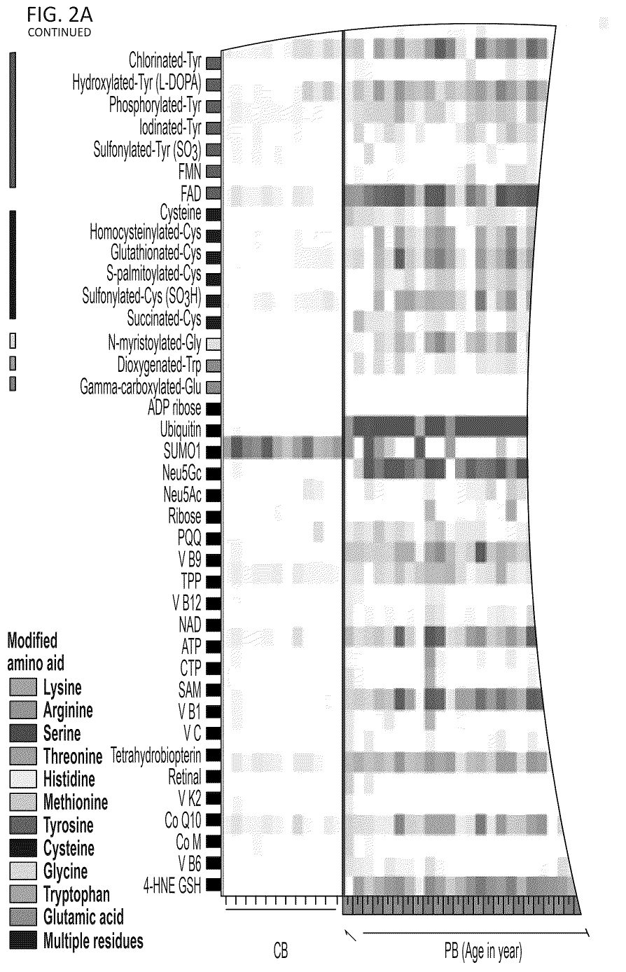

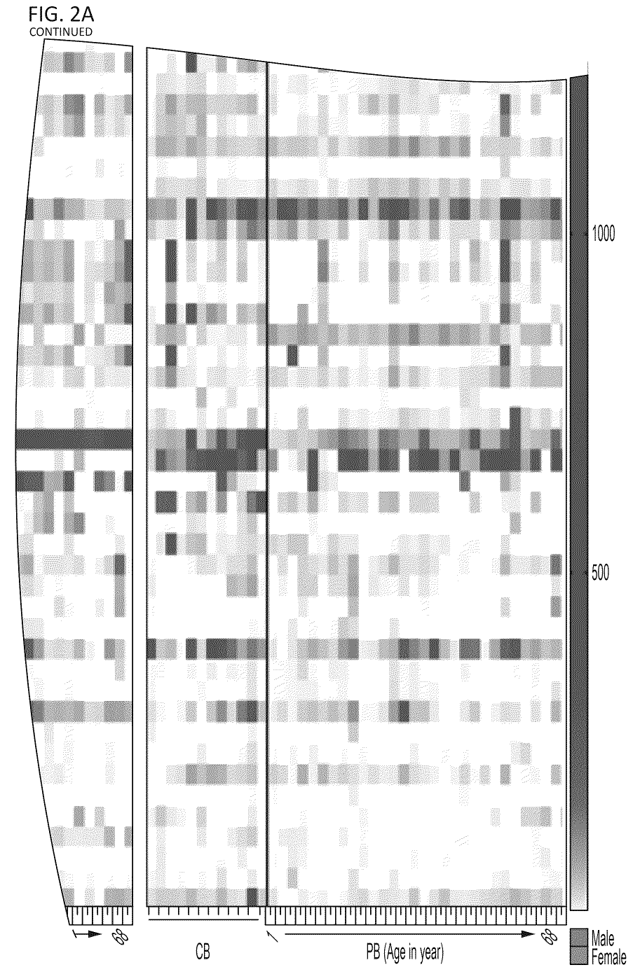

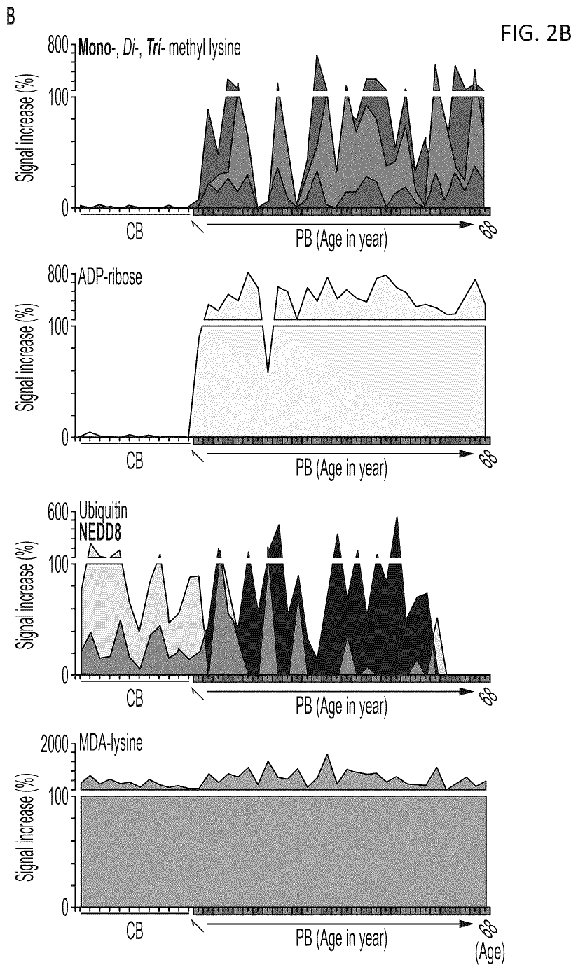

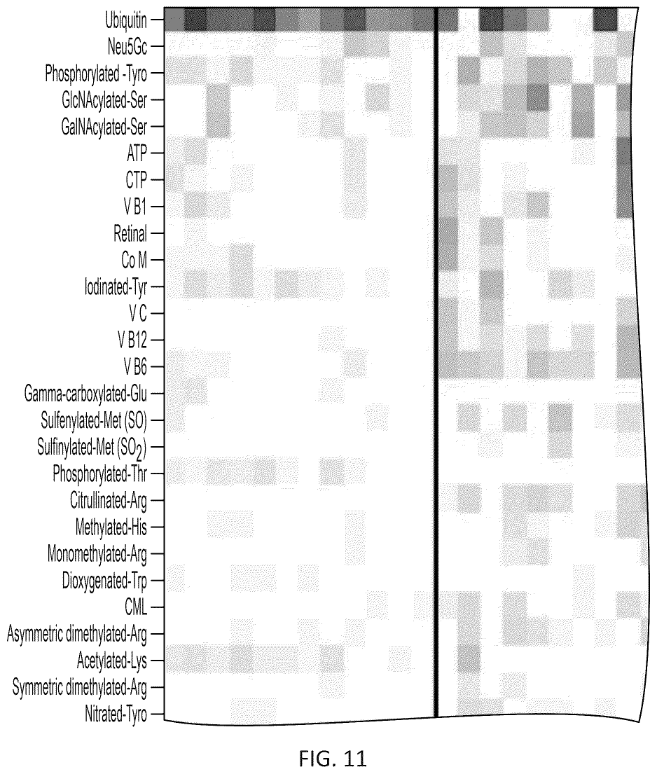

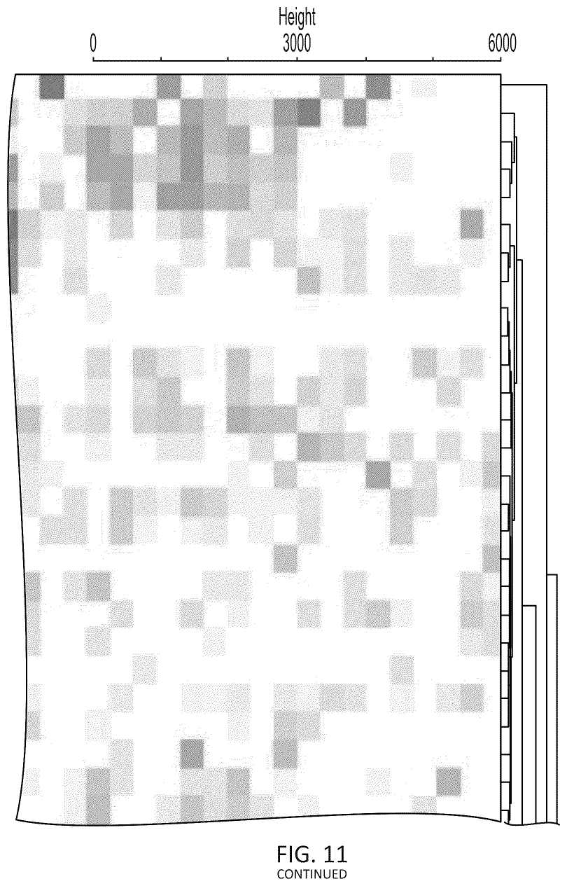

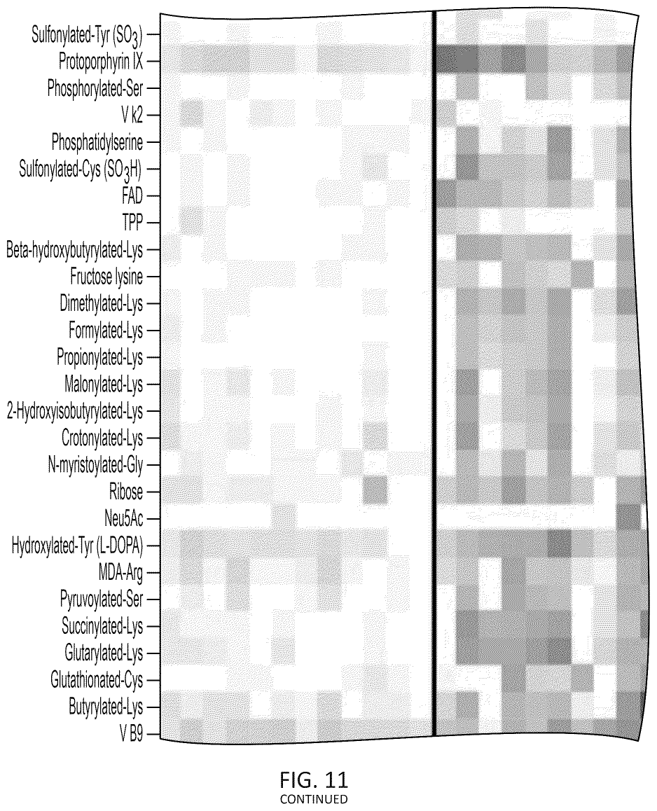

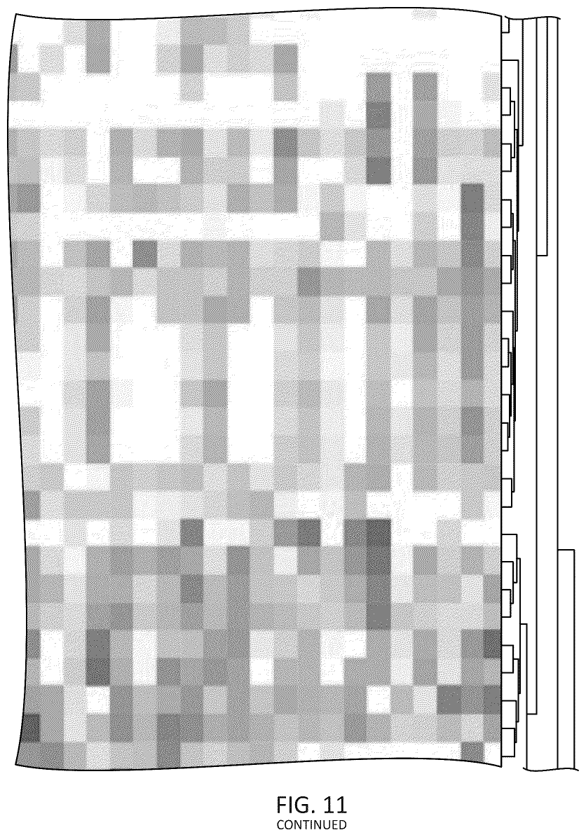

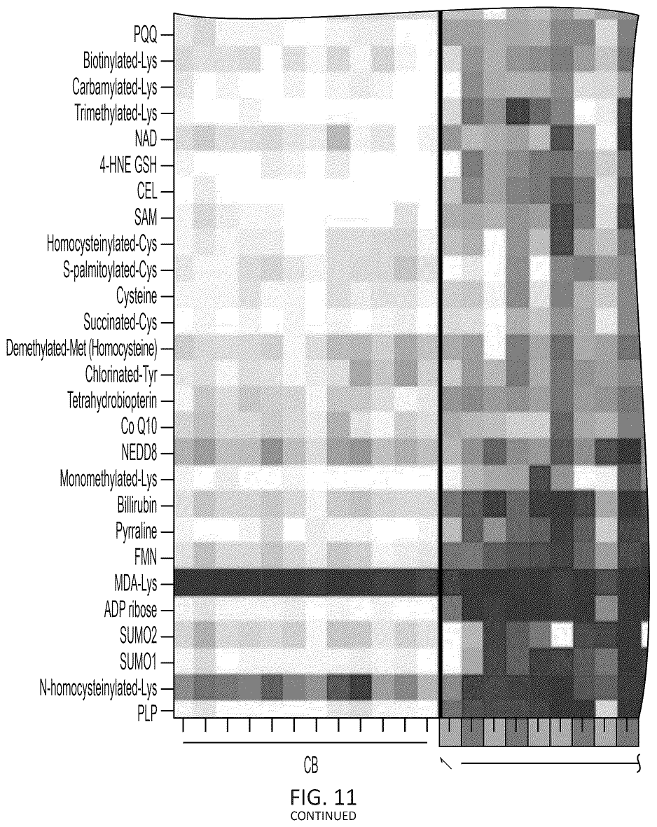

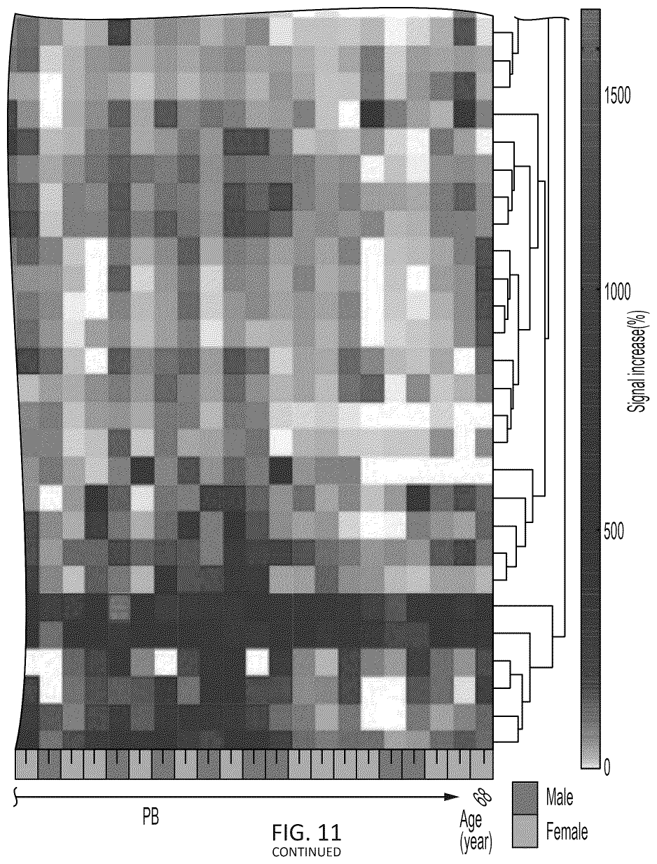

The presence of memory B cells reacting to multiple adducts in the peripheral blood of healthy donors suggested that the serum antibody repertoire encompassed these and other adduct specificities. To test this hypothesis, we assessed the reactivity of IgM and IgG in cord blood (CB) samples obtained from 12 newborns and peripheral blood (PB) specimens collected from 30 healthy subjects ranging from 1 to 68 years of age to all 81 adducts included in our panel. As shown in FIG. 2A, and FIG. 11, serological profiling revealed a complex pattern of reactivity to a wide range of adducts with important disparities between IgM and IgG. Several adducts were recognized by most subjects (e.g. MDA), while others were only recognized by some individuals or barely recognized. At the population level, no major differences were noticeable between anti-adduct reactivity of CB and PB IgG, an expected observation in keeping with the active transplacental transfer of maternal IgG to the fetus²⁰. In contrast, the reactivity of CB IgM, which originated exclusively from the fetus, markedly differed from that of PB IgM of 1 to 68 year-old donors. Four adducts, N-homocysteine, MDA-Lysine, NEDD8 and ubiquitin, dominated CB IgM profiles. Neddylation and ubiquitylation are two essential PTM involved in cell growth, apoptosis and embryogenesis^{21, 22}. N-homocysteinylation is linked to oxidative stress and MDA has long been identified as a critical oxidation-specific epitope (OSE) exposed on apoptotic cells and damaged self-structures^{23, 24}. Abundance of molecules bearing these 4 adducts during fetal life may have triggered the production of specific IgM. On the other hand, IgM reactive to other ubiquitous adducts were virtually undetectable in CB but rapidly developed after birth as illustrated in FIG. 2B for mono-, di- and trimethyl-lysine as well as ADP-ribose. Serum IgM reactivity to NEDD8 and ubiquitin fluctuated after birth whereas IgM reactivity to MDA-lysine remained steady (FIG. 2B).

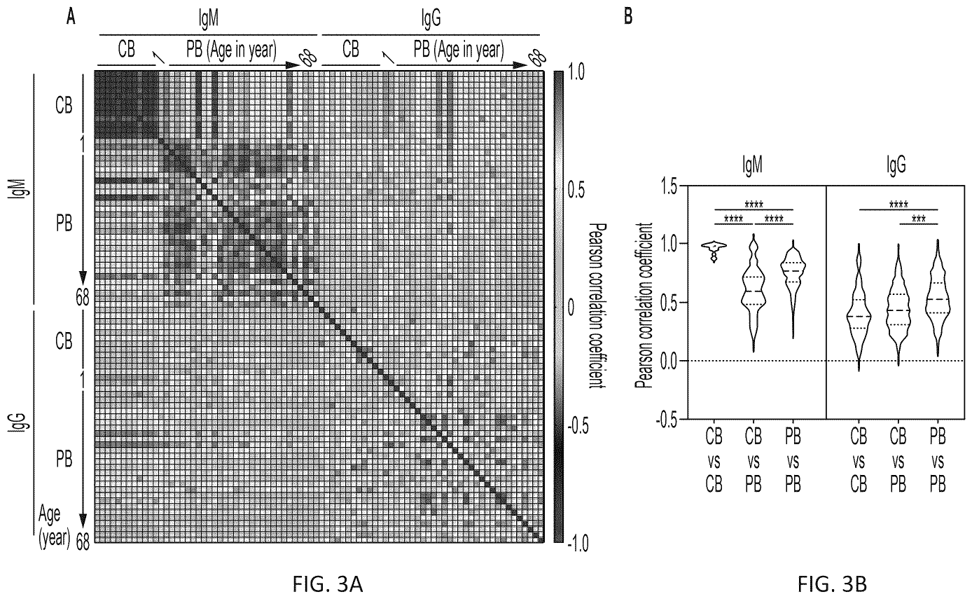

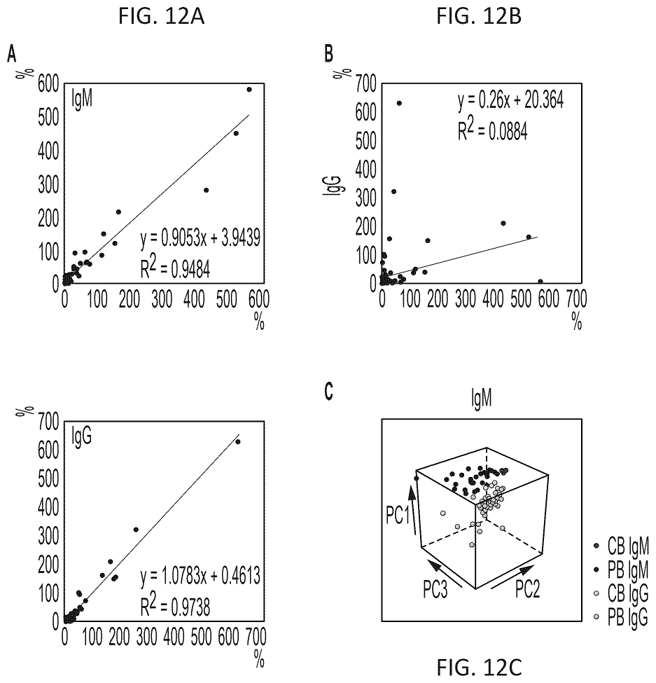

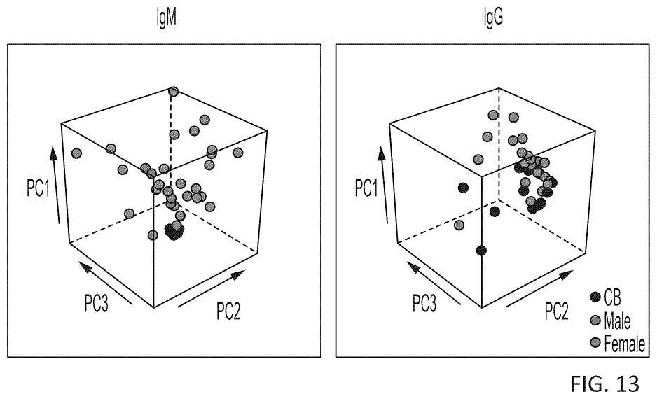

A pairwise correlation analysis further confirmed the nearly identical reactivity profiles of IgM present in all CB specimens (FIGS. 3A and B). These markedly diverged from IgM profiles of PB collected from 1 to 68 year-old donors. Moreover, the anti-adduct IgM reactivity pattern appears remarkably constant after birth and across the lifespan with limited variations between subjects. Such unexpected consistency implies that a robust mechanism is responsible for the regulation of IgM humoral immunity to common adducts and suggests important functional properties. Contrasting with IgM, anti-adduct IgG profiles were highly diverse between individuals, likely reflecting each individual's immunological history (FIGS. 3A and B). Furthermore, little correlation was detected between IgM and IgG reactivity patterns in both CB and PB (FIG. 3A and FIG. 12), setting apart these two distinct components of humoral immunity. Lastly, while limited in size, this analysis did not reveal any noticeable difference in anti-adduct IgM and IgG reactivity between men and women (FIG. 13).

### The Anti-Adduct IgM Repertoire Abruptly Diversifies During Infancy

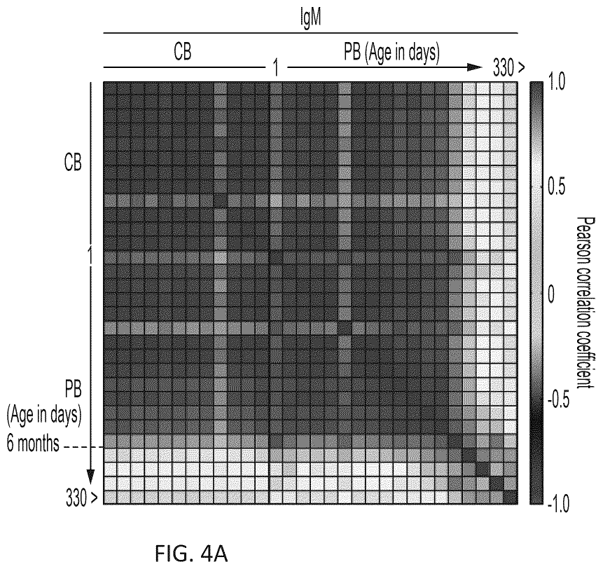

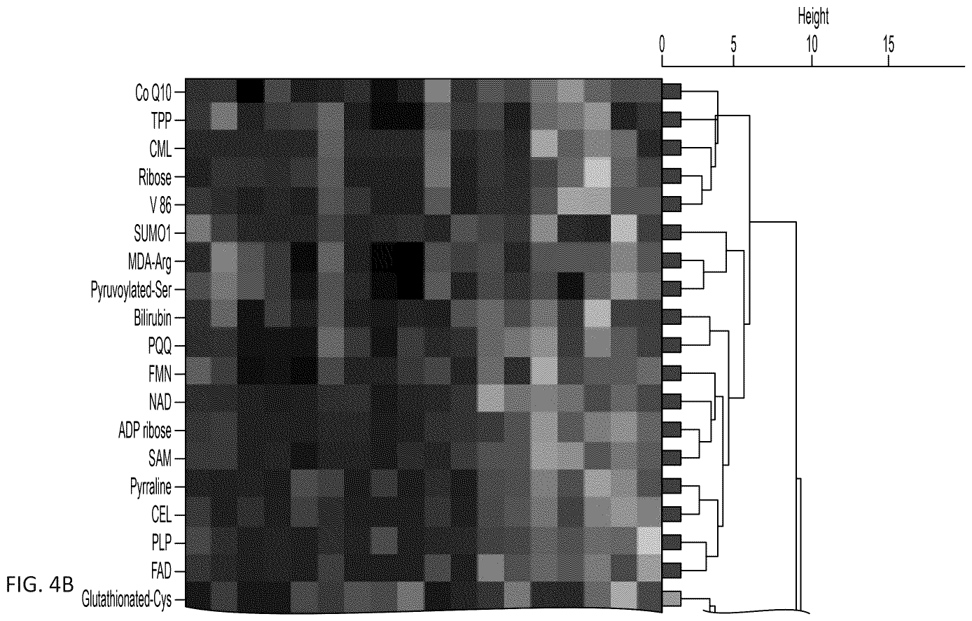

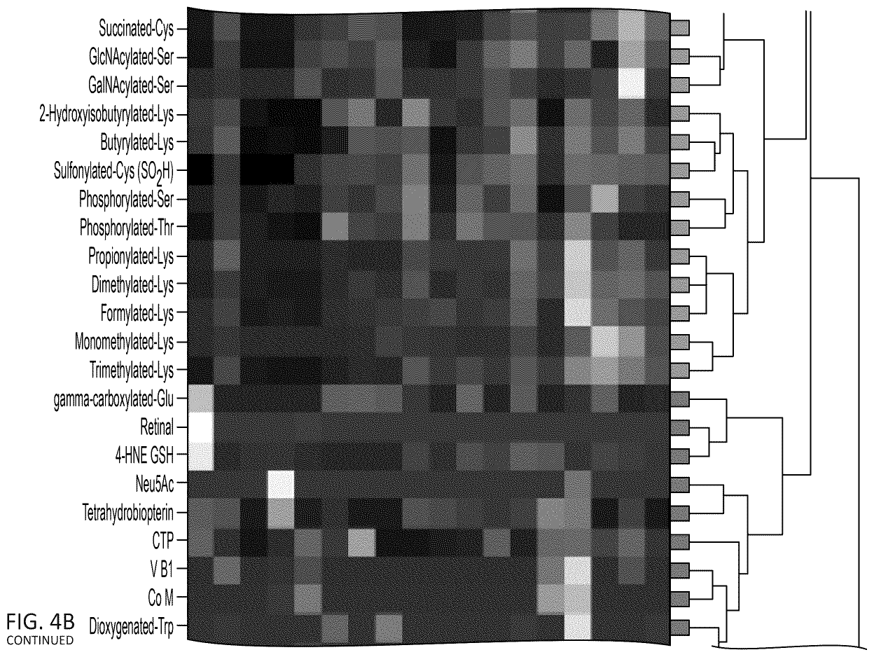

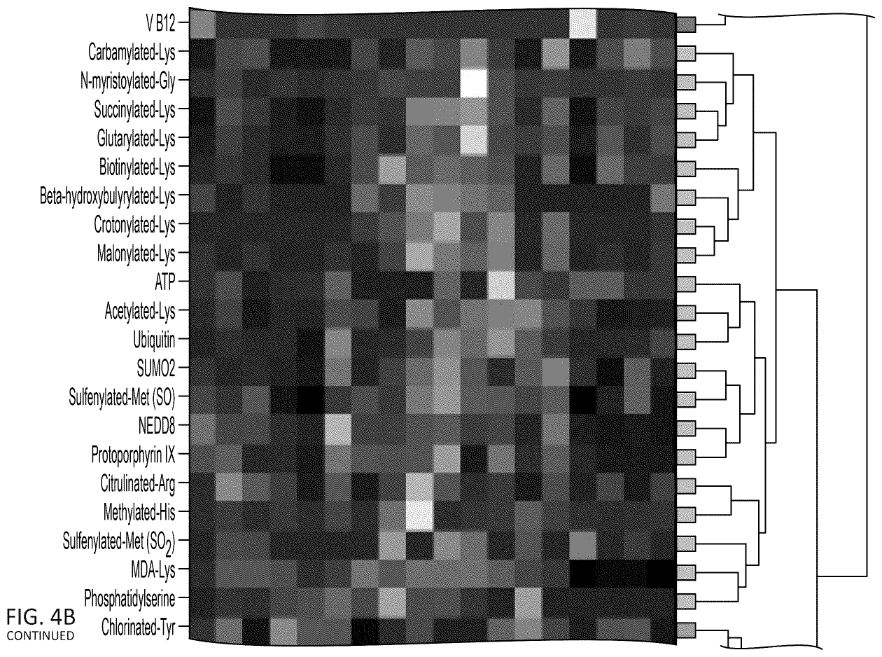

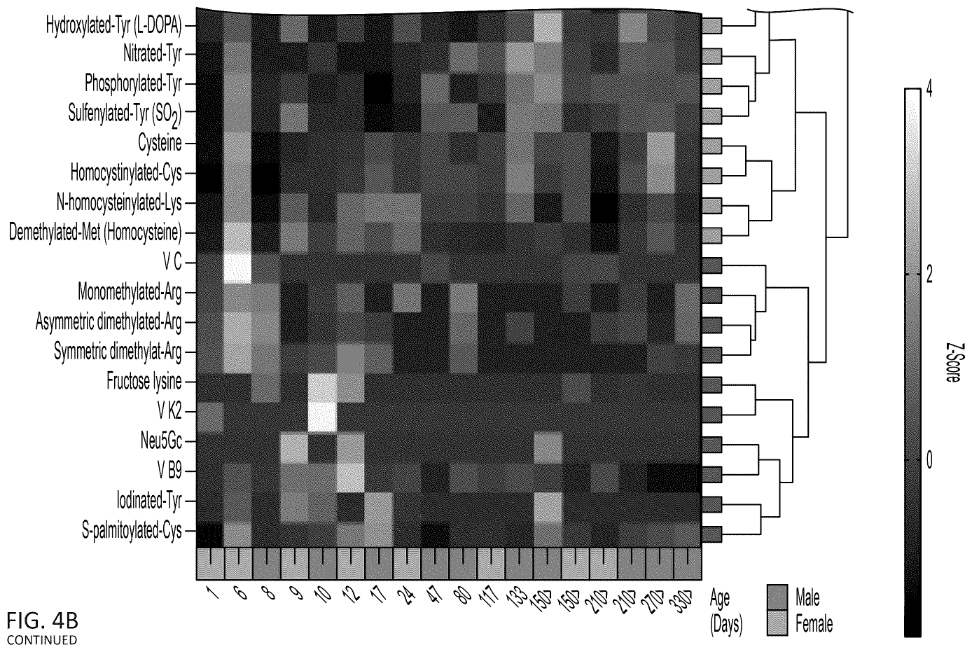

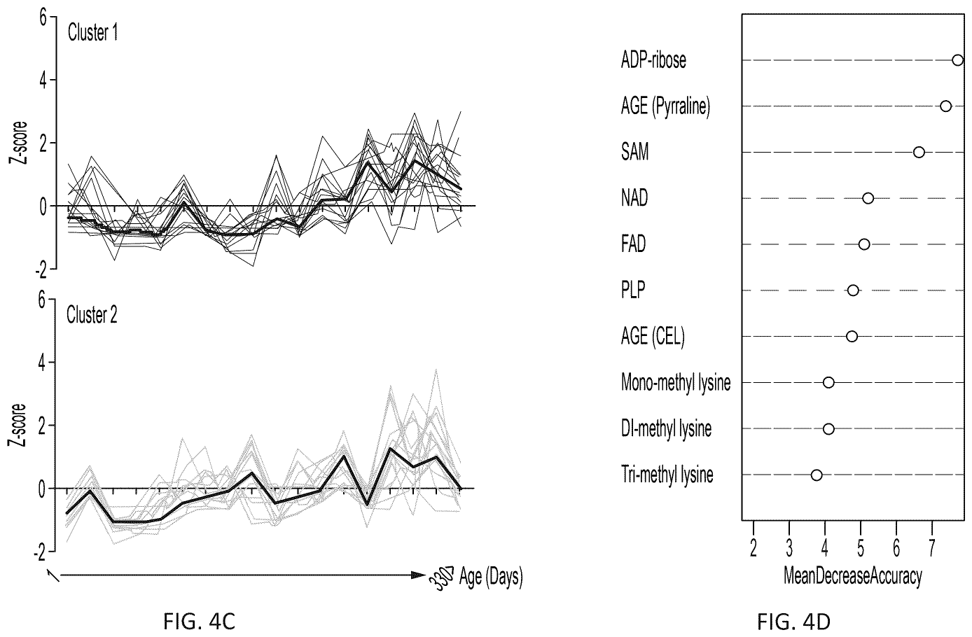

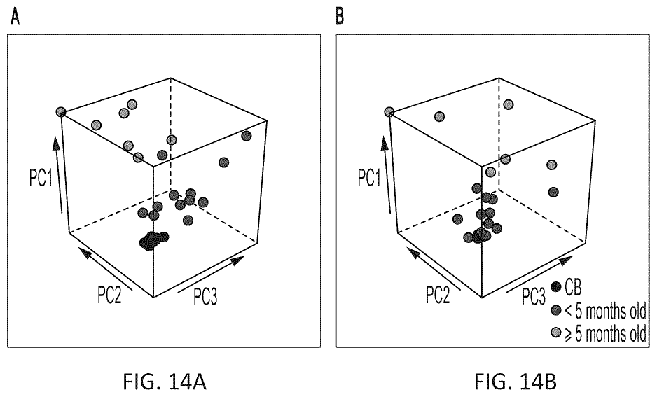

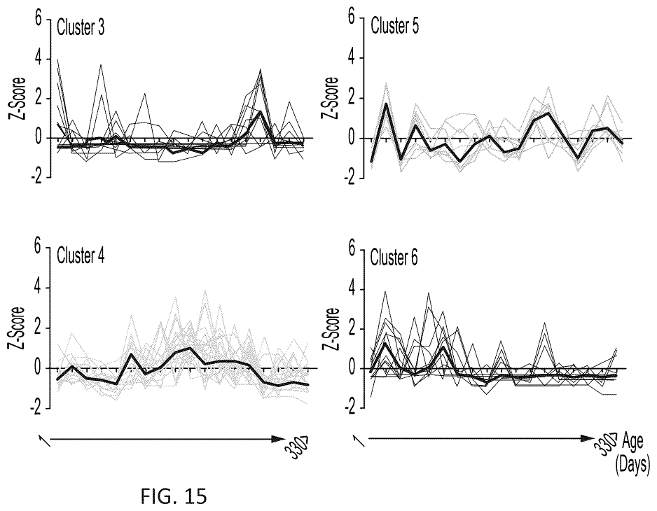

To further investigate the abrupt change in serological reactivity during early life, we assessed anti-adduct IgM in the blood of 18 infants aged 1 day to 11 months. A correlation matrix revealed a sudden transition in serum IgM reactivity, occurring approximately at 6 months of age (FIG. 4A and FIG. 14). Unsupervised clustering of normalized reactivity values distinguished those adducts towards which the most IgM developed during the first year of life (FIG. 4B). Several temporally restricted clusters were apparent in this analysis. Yet, two clusters (cluster 1, blue, and cluster 2, grey, in FIG. 4B) comprised 32 adducts targeted during the early life immune conversion observed in our correlation matrix (FIG. 4A). Serum reactivity to these adducts increased sharply after 6 months (FIG. 4C), whereas reactivity to other adducts was either unchanged or fluctuated inconsistently (FIG. 15). A random forest classifier, using the Boruta algorithm, identified the top 10 most discriminative adducts separating neonatal from adult IgM profiles (FIG. 4D). Among these top 10 targets, were four PTM (ADP-ribose, mono-, di- and tri-methyl lysine), three essential co-enzymes, S-adenosyl-methionine (SAM), nicotinamide adenine dinucleotide (NAD) and flavin adenine dinucleotide, (FAD), two advanced glycation end products (AGE)^{25, 26}, pyrraline and carboxyethyl-lysine (CEL) as well as an AGE inhibitor, pyridoxal phosphate (PLP)²⁷.

### Endogenous Adducts Contribute to Shaping the IgM Repertoire in Early Infancy

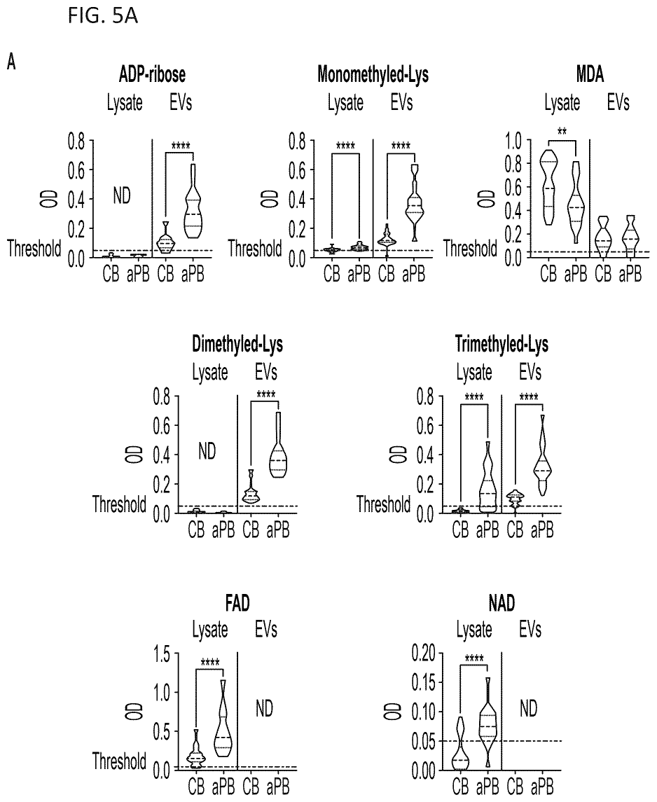

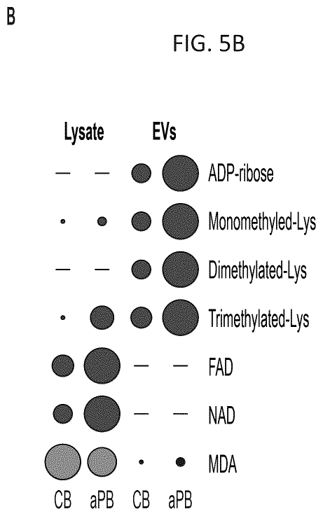

The sudden wave of change in IgM immunity to natural adducts during early life can be partly explained by exposure to multiple environmental antigens. On the one hand, development of the microbiome in infants is accompanied by antibody responses to bacterial antigens that include ubiquitous adducts^{28, 29, 30, 31}. For instance, N-acetyl-glucosamine (GlcNac) exposed on bacterial cell walls triggers robust humoral immunity in young mice³². A similar mechanism could result in anti-adduct antibodies in humans. Remarkably, GlcNac was among the 32 adducts towards which IgM developed the most after 6 months of age (Grey cluster 2 in FIG. 4B). Dietary antigens form another category of exogenous antigens that human infants are exposed to^{33, 34}. It is noteworthy that two AGE and one AGE inhibitor are among the 10 most informative adducts, discriminating IgM reactivity profiles of neonates and infants up 6 months of age from older infants, children and adults (FIG. 4D). AGE products result from chemical transformation of amino acids and sugars such as that resulting from the Maillard reaction during the cooking process²⁶. Our findings therefore suggest a contribution of food antigens to the development of anti-adducts immunity in infants. Aside from exogenous antigens, we hypothesize that exposure to endogenous antigens in the form of modified proteins and macromolecules may also contribute to anti-adduct antibody responses. Modifications of proteins, lipids and nucleic acids are abundant in humans, some of which are well-described epigenetic changes involved in aging^{35, 35, 36, 37}. We investigated whether accumulation of several such modifications, starting during the first year of life, could have elicited specific IgM responses and helped shape the adult anti-adduct repertoire. To test this hypothesis, we measured the level of the 10 most discriminative adducts (FIG. 4 D) in peripheral blood mononuclear cells (PBMC) and extracellular vesicles (EVs) isolated from CB (N=25) or adult PB (aPB, N=25) as a marker of their overall abundance at two different stages of life. We also assessed the level of MDA-lysine as a control adduct towards which IgM reactivity does not change significantly from birth to adulthood (FIGS. 2A and B). CEL, pyrraline, PLP and SAM were undetectable. However, the 6 remaining adducts were detected in either PBMC lysate, EVs or both. As shown in FIG. 5, their levels were significantly higher in aPB than in CB. The overall level of control MDA-lysine, however, was lower in aPB than in CB. These results support the development of specific IgM in response to modified self in the form of epitopes affixed to proteins or other macromolecules. This mechanism is comparable to the generation of antibodies to citrulline adducts in patients with rheumatoid arthritis triggered by the accumulation of citrullinated proteins in the joints^{38, 39}.

### Discussion of Example 1

A profuse yet scattered literature reports the existence of antibodies to adducts composed of chemical groups attached to macromolecules. These include post-translational modifications, metabolites, co-enzymes, carcinogenic and pollutant byproducts linked to DNA⁴⁰. Lysophosphatidylcholine is an example of such an adduct exposed on apoptotic cells and recognized by natural IgM⁴¹. Pathogenic antibodies to specific adducts have also been described in the context of several diseases. For instance, IgG to citrullinated proteins are both prognostic and predictive of rheumatoid arthritis³⁹. Systemic lupus erythematosus associates with antibodies to numerous phosphorylated antigens⁴². Moreover, antibodies reactive to MDA contribute to the pathophysiology of atherosclerosis^{23, 43}. Aside from these isolated cases, however, the overall humoral immunity to naturally occurring adducts had not been thoroughly examined. Here we provide evidence of dynamic serological responses to a wide range of adducts in humans, starting during fetal life and maintained across the lifespan. Even if our panel only covers a sample of all existing adducts, our findings reveal a complex repertoire, including several unreported specificities.

The study also provides an alternate explanation for polyreactivity, a common pattern observed for ~20% of human peripheral blood mature naïve B cells and a majority of circulating IgG+ switched memory B cells^{12, 13, 14}. We propose that recognition of single motifs such as phosphoryl or methyl groups, exposed on different macromolecules, results in apparent polyreactive profiles. Following this hypothesis, a large fraction of B cells and antibodies initially characterized as polyreactive may in fact react to specific adducts. This novel view also contradicts the general understanding that polyreactive Nabs form a homogeneous class of antibodies. In reality, the anti-adduct repertoire may be equally diverse as the repertoire of antibodies specific to protein epitopes.

The broad and steady IgM immunity to adducts throughout life suggests the existence of a persistent pool of unswitched memory B cells maintaining the repertoire. Among the candidates are marginal zone (MZ) B cells often considered part of a loosely defined pool of innate-like B cells in humans or B1 B cells in mice^{11, 44, 45}. A main characteristic of innate-like B cells is their ability to secrete antibodies with a polyreactive profile commonly referred to as Nabs^{1, 2, 3, 46, 47} Circulating IgM+ memory B cells, previously described as a distinct subset sharing a number of features with MZ B cells⁴⁸, could also participate in maintaining the Nabs pool. As explained above, because the appearance of polyreactivity, may in some instances, be explained by the specific recognition of a single adduct exposed on multiple macromolecules, we reason that a significant proportion of Nabs could correspond to anti-adduct antibodies.

Another intriguing observation resulting from the investigation is the striking differences between anti-adduct IgM and IgG profiles throughout life. Antibody responses are conventionally thought to unfold through sequential waves, starting with the generation of IgM followed by the successive development of IgG subclasses binding to the same immunogen with increased affinity. However, the findings herein reveal limited correspondence between IgM and IgG profiles suggesting that at least with respect to anti-adduct responses, these two arms of humoral immunity may target different antigens. The higher interindividual correlation obtained for IgM reactivity than for IgG reactivity is also particularly interesting. Several explanations can be considered to account for this observation: 1) the IgM repertoire may be maintained across the lifespan by constant stimulation with the same set of unchanging antigens, such as self-antigens, whereas the IgG repertoire may be primarily shaped by the encounter with highly diverse antigens such as those found on pathogens; 2) the clonal composition of the IgM-secreting cell subset may vary less than that of the IgG-secreting cell subset. The latter may be more prone to robust clonal expansion, affecting the IgG repertoire, 3) genes coding for IgM usually harbor less somatic mutations than those coding for IgG. The overall IgM repertoire may therefore be closer to a “germline” repertoire shared among individuals and consequently more stable across the lifespan.

The study revealed the unappreciated scale and dynamics of antibody responses to natural adducts across the lifespan. Focusing on IgM, we uncovered that this humoral immunity abruptly diversifies around 6 months of age, marking the transition from a neonatal to an adult repertoire. Two main types of antigens can account for the development of anti-adduct antibodies first in utero and then during early life when diversification occurs: exogeneous, i.e. environmental antigens, or endogeneous antigens. As previously reported, exposure to microbiota and additional environmental antigens during the first year is a probable factor in this transition^{28, 29, 30, 31}. The sharp increase in IgM reactivity to two AGE after 6 months of age is also noteworthy as it suggests a role for food antigens in this reaction. Regarding endogenous antigens, our findings suggest that epigenic modifications such as methyl and ADP-ribose, accumulating after birth through the aging process, may also constitute a source of naturally occurring adducts and contribute to modelling the IgM repertoire. The recognition that self-adducts elicit robust antibody responses has important implications beyond the development of immunity in early life. Responses to specific adducts can also reflect abnormal modifications associated with certain diseases as illustrated by the generation of anti-citrulline adduct antibodies in RA³⁹. Interrogating the anti-adduct repertoire could therefore be utilized as a means to identify concealed modifications associated with pathological conditions through the detection of specific antibodies.

The findings herein represents the first mapping of the anti-adduct antibody repertoire in humans from birth to old age. Results reveal a complex and dynamic profile evolving with age. Collectively, these findings provide the conceptual and methodological framework to further investigate this underreported component of humoral immunity and determine its homeostatic function and possible implication in multiple pathologies.

### Methods for Example 1

### Human Specimens

Cord blood samples were obtained from the Carolinas Cord Blood Bank (Duke University, Durham, NC). Plasma specimens collected from 1 to 68-years-old donors and serum specimens from 1 day to 330 days-old infants were purchased from Lee Biosolutions, Inc (Maryland Heights, MO). Buffy coats from healthy adults were obtained from the New York Blood Center and used for adduct level measurements. Plasma samples from 20 to 73-years-old healthy donors used to extract microvesicles were purchased from Innovative Research, Inc. (Novi, MI USA). All samples were discarded and de-identified according to the Protected Health Information (PHI) regulations and considered non-human research samples. This study was approved by the Columbia University Institutional Review Board. All sample information was listed in extended data tables 2 to 4.

### Adduct Panel

We designed and ELISA panel of 81 adducts and controls as follows: A) 46 synthetic 5-mer peptides, each composed of a modified amino acid flanked by 4 unmodified arginine; 5-mer peptides with matching unmodified residues were included in the panel as controls, B) 6 modified amino acids, C) 8 PTM compounds (e.g. SUMO, ubiquitin) and D) 21 co-factors, co-enzymes and metabolites (e.g. coenzyme Q10). The panel also includes MDA-BSA, insulin and LPS as positive controls. MDA-modified BSA and MDA-Lysine/Arginine peptides were generated by incubating acid-hydrolyzed 1,1,3,3-tetramethoxypropane (Sigma-Aldrich, St. Louis, MO) with BSA or Lysine/Arginine peptides. Briefly, 2 M 1,1,3,3-tetramethoxypropane was hydrolyzed in 96 mM HCl for 15 minutes at 37 °C and then neutralized with NaOH. BSA (2 mg/ml) or Lysine/Arginine peptides (10 mg/mL) with 0.2 M MDA was incubated for 3 hours at 37 °C. Extensive dialysis against PBS 1× was performed for MDA-BSA at 4 °C for 36 hours. All adducts and controls included in the panel are detailed in extended data table 1, together with their sources and working concentrations.

### Detection of Adduct-Reactive Antibodies

IgM and IgG reactivity to the 81 adducts included in the panel was quantified by ELISA as follows. Nunc MaxiSorp™ high protein-binding capacity 96 well ELISA plates (Thermo Fisher Scientific) were coated in duplicate with adducts, compounds and control antigens at 4 °C for 20 hours. Synthetic peptides with modified amino acids, large PTM compounds and small compounds were coated at 10 μM, 1 μM and 1 mM respectively (Table 2). ELISA plates were washed three times in PBST and then blocked with 3% BSA (Fisher Scientific Inc.) in PBST for 2 hours at 37 °C. Serum or plasma diluted in PBS at 1:50 for IgM and 1:200 for IgG was distributed to the plate and incubated for 3 hours at room temperature. Plates were then washed five times in PBST and incubated with horseradish peroxidase (HRP) conjugated anti-human IgM or IgG affinity-purified F(ab′)₂ fragment (Jackson ImmunoResearch Laboratories, Inc., West Grove, PA) diluted in blocking buffer for 1 hour. Plates were then washed again and HRP activity was developed with 3,3′,5,5′-tetramethylbenzidine (TMB, Fisher Scientific Inc.). All reagents used in ELISA are listed in extended data table 5. Reactivity to adducts was calculated and reported as signal increase (%) over signal obtained with controls. For synthetic peptides with modified AA, matching peptides with unmodified AA were used as controls. For other compounds, signal was compared to blank. Normalization was conducted using reactivity to LPS, insulin and MDA-BSA as internal controls. Standardization, unsupervised clustering, random forest classification with the Boruta feature selection of signal increase values was performed using R version studio version 3.6.2. Pearson correlation coefficients and normalization values were calculated using GraphPad Prism 9.

### Measurement of Adduct Levels

Adduct levels were measured in lysates prepared from PBMC as well as in extracellular vesicles (EV) isolated from CB and adult peripheral blood from healthy donors. Lysate was prepared with RIPA buffer (Boston Bioproducts, Inc. Ashland, MA) containing EDTA-free protease inhibitor (Roche). Lysates were clarified by centrifugation at 12000×g at 4 °C for 15 minutes before adduct level detection. EV were extracted from each CB plasma and adult donor plasma using the total Exosome Isolation Kit (Thermo Fisher Scientific). Protein concentration was determined in cell lysate and EV using the BCA Protein Assay kit (Thermo Fisher Scientific) and adjusted to coat equal amounts for each sample on Nunc MaxiSorp™ high protein-binding capacity 96 well ELISA plates (Thermo Fisher Scientific) at 4 °C for 18 hours for adduct detection by ELISA. The following antibodies were used for specific adduct detection: Mono-Methyl Lysine [mme-K] MultiMab™ Rabbit mAb, Di-Methyl Lysine [dme-K] MultiMab™ Rabbit mAb, Tri-Methyl Lysine [tme-K] (D1L1X) Rabbit mAb, Poly/Mono-ADP Ribose (E6F6A) Rabbit mAb (Cell Signaling Technology, Inc., Danvers, MA). Antibody binding was then revealed using HRP conjugated Goat Anti-Rabbit IgG (abcam, Cambridge, MA). For the quantification of FAD, NAD and MDA the following kits were utilized: Flavin Adenine Dinucleotide Assay Kit (ab204710) and NAD/NADH Assay Kit (ab65348) (abcam, Cambridge, MA) and OxiSelect™ MDA Adduct Competitive ELISA Kit (STA-832-5) (Cell Biolabs, Inc., San Diego, CA).

### Isolation and Immortalization of B Cell Clones

The procedure for isolation and immortalization of B cell clones has already been described⁴⁹. In brief, CD20+CD27+ memory B cells were isolated by cell sorting from the PBMC of healthy adult donors then immortalized by incubation with supernatant from Epstein-Barr virus-producing B95-8 marmoset cells in presence of the TLR9 agonist CpG 2006 at 2.5 μg/ml. All transformed clones were generated by limiting dilution. Clonality was confirmed by molecular analysis of Ig heavy chain transcripts as described⁴⁹.

### Immortalized B Cell Clone Heavy Chain Variable Region Analysis

Total RNA was extracted from immortalized clones D8C6, D9B9, and M2.3 using TRIzol reagent (Thermo Fisher Scientific) and cDNA was synthesized using Superscript III reverse transcriptase kit (Thermo Fisher Scientific) as described⁴⁹. Variable regions of the Ig heavy chain were amplified by PCR using 6 family specific forward primers (VH1 to VH6) and a consensus JH reverse primer. The PCR conditions were as follows: 95 °C/5′, (95 °C/30″; 56 °C/30″; 72 °C/30″)×35 cycles; 72 °C/10′. PCR products were cloned in a pCR8/GW/TOPO TA vector (Thermo Fisher Scientific) and used to transform TOP10 chemically competent bacteria (Thermo Fisher Scientific). Twenty representative colonies per clone were harvested and sequenced using the corresponding VH primer to confirm the clonality of the B cells.

### Monoclonal IgM Reactivity to Apoptotic Cells

Viable Jurkat cells were UV irradiated using a stratalinker 2400 (LabX, Midland, ON, Canada) and incubated overnight. Apoptotic cells suspension was next stained with IgM mab secreted by immortalizing memory B cell clones derived from healthy adult donors. Staining was revealed using Brillant Violet™ 421 anti-human IgM (MHM-88). Apoptotic Jurkat cells were co-stained with 7-AAD Viability and FITC or APC Annexin V (BioLegend). Stained cells were analyzed by using FACS LSRFortessa™ (Becton Dickinson) with FCS express 7 flow cytometry analysis software. Antibodies used in flow cytometry are listed in extended data table 5.

Monoclonal IgM Reactivity to Insulin, MDA-BSA, dsDNA, LPS, MDA and Phosphoryl Groups and Competitive ELISA

Monoclonal IgM reactivity to generic self-antigens was tested as previously described⁵⁰. Briefly, Insulin (Sigma-Aldrich, St. Louis, MO), MDA-BSA, dsDNA (Sigma-Aldrich, St. Louis, MO) and LPS (Millipore Sigma, Burlington, MA) were coated on Nunc MaxiSorp™ high protein binding capacity 96 well ELISA plates (Thermo Fisher Scientific) in duplicate at 4 °C for 20 hours. Plates were washed three times in PBST and then blocked with 3% BSA (Fisher Scientific Inc.) in PBST for 2 hours at 37 °C. Monoclonal IgM at a concentration of 2.5 μg/ml diluted in PBS was distributed to the plate and incubated for 3 hours at room temperature. Plates were then washed five times in PBST and incubated with horseradish peroxidase (HRP) conjugated anti-human IgM affinity-purified F(ab′)₂ fragment (Jackson ImmunoResearch Laboratories, Inc.) diluted in blocking buffer for 1 hour. Plates were then washed again and HRP activity was developed with 3,3′,5,5′-tetramethylbenzidine (TMB) (Fisher Scientific Inc.). For competitive ELISA, the monoclonal IgG reactivity to insulin and LPS was assessed either alone or in the presence of phosphorylated-threonine peptide at a final concentration of 10 μM. Reactivity to MDA and phosphoryl groups on insulin, LPS and dsDNA was tested using the same method with an anti-MDA mouse monoclonal IgG1 (MA5-27559), Fisher Scientific Inc.) and an anti-phosphoserine/threonine/tyrosine rabbit IgG (61-8300 Fisher Scientific Inc.).

### Immunoprofiling Using Protein Microarrays

Monoclonal IgM reactivity profiling was performed using V4.0 protoarrays (Thermo Fisher Scientific) as previously described⁵¹ except for the following: Arrays were probed with immortalized clone culture supernatant containing monoclonal antibody at a concentration of 5 μg/ml of IgM in probing buffer. The reactivity to the 8027 proteins was compared for each monoclonal antibody to the one obtained after probing an array with the secondary anti-IgM antibody alone, which represents the background signal. We considered positive a signal which was at least 5 times higher than the background with a difference in signal equal or greater than 100.

### Statistical Analysis

Unpaired t-test or Mann-Whitney test were performed following Shapiro-Wilk normality test with GraphPad Prism 9. ** P<. 01 and **** P<. 0001.

### Example 2: Multiplex Detection of Adducts

According to another embodiment, a biological sample provided from one or more subjects is analyzed for the presence or absence of natural antibodies reactive to one or more adducts as set forth in Tables 1A and 1B (supra) utilizing multiplex assay. The multiplex assay involves:
1. Attaching each adduct selected to a plurality of microspheres with a distinct fluorometric signature. This attachment may be direct or indirect;
2. Pooling the adduct/microspheres to create a adduct/multiplex reagent, such as for a kit;
The adduct/microsphere reagent can then be used for analyzing as follows:
3. Mixing a biological sample with the adduct/microsphere reagent;
4. Washing the mixture to remove unbound biological sample;
5. Detecting natural antibody(ies) from the biological sample that is/are captured by an adduct or multiple adducts detected using a fluorescently labeled reagent that binds to the natural antibody. In a specific example, the fluorescent label used s Phycoerythrin

### Example 3: Serological Responses to Tumor-Associated Adducts in Pancreatic Cancer

The incidence of pancreatic cancer has increased over the past several decades. This cancer is now the fourth leading cause of cancer death in the USA. Less than 10% of the patients live beyond 5 years after their initial diagnosis. This dismal survival rate is partly explained by the fact that most pancreatic cancer cases are detected late when treatment options are limited. It has long been recognized that cancer cells abnormally accumulate chemical groups covalently linked to proteins, DNA and other molecules. Serological responses to these “adducts” have not been thoroughly explored.

Similar to that described in Example 1, ELISA platform has been developed for the detection of serum IgG reactive to 93 adducts (adducts enumerated in Tables 1A and 1B), including post-translational modifications known to contribute to tumorigenesis, progression and metastasis. The panel also includes oxidation-related modification, advanced glycation end products, and certain co-enzymes that qualify as adducts based on their binding properties. Using this assay, anti-adduct antibodies in the plasma of adult healthy donors (N=40; average age 56) as well as patients with pancreatic cancer (N=39; average age 68) were measured. All patient specimens were collected before any treatment.

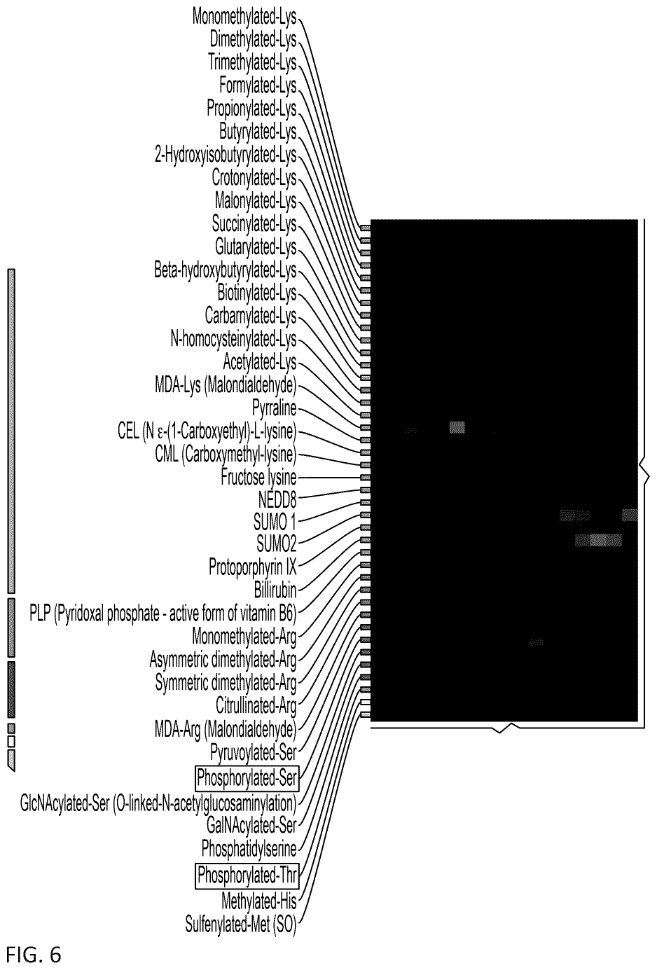

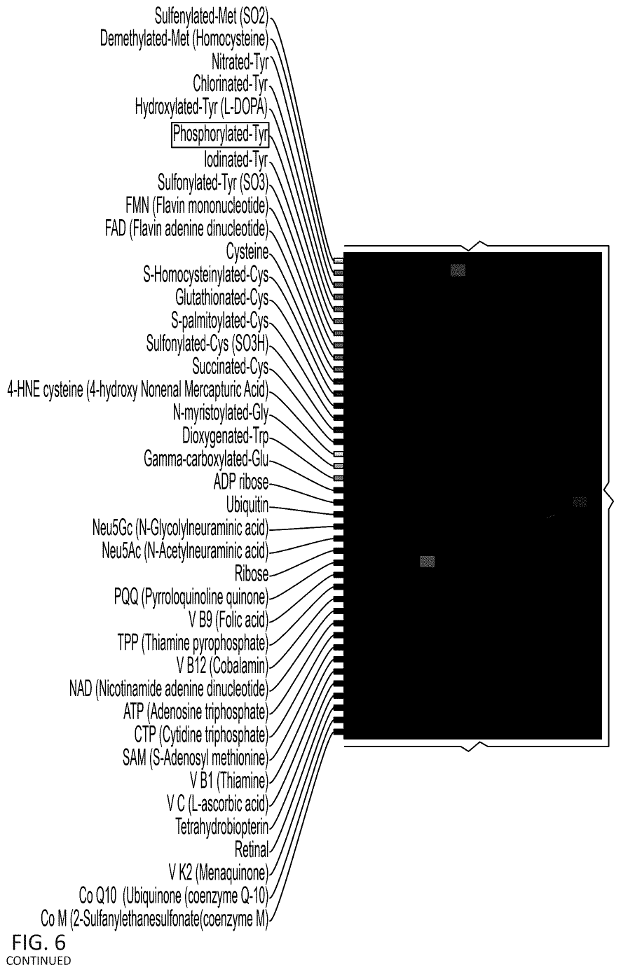

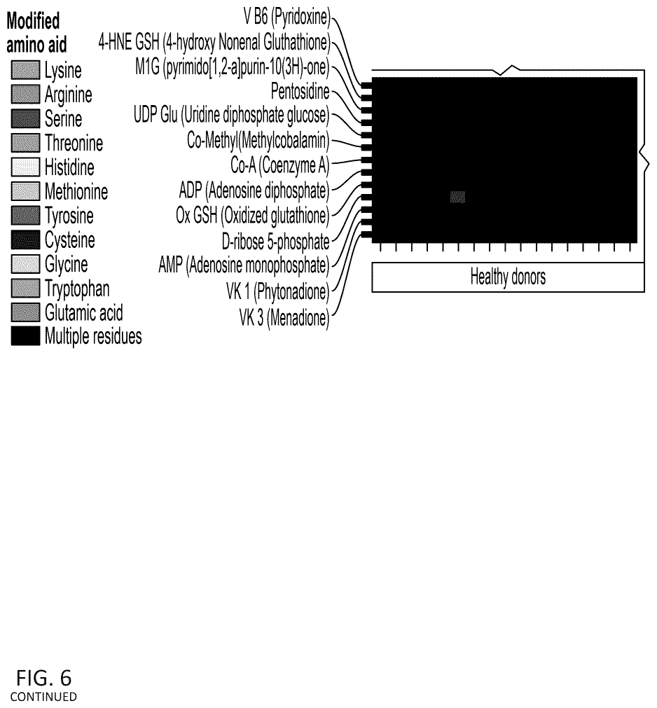

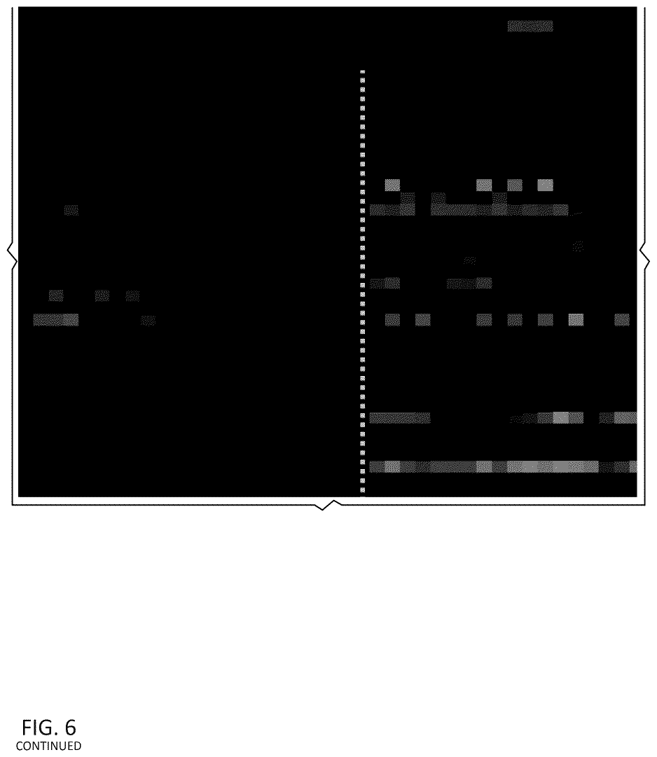

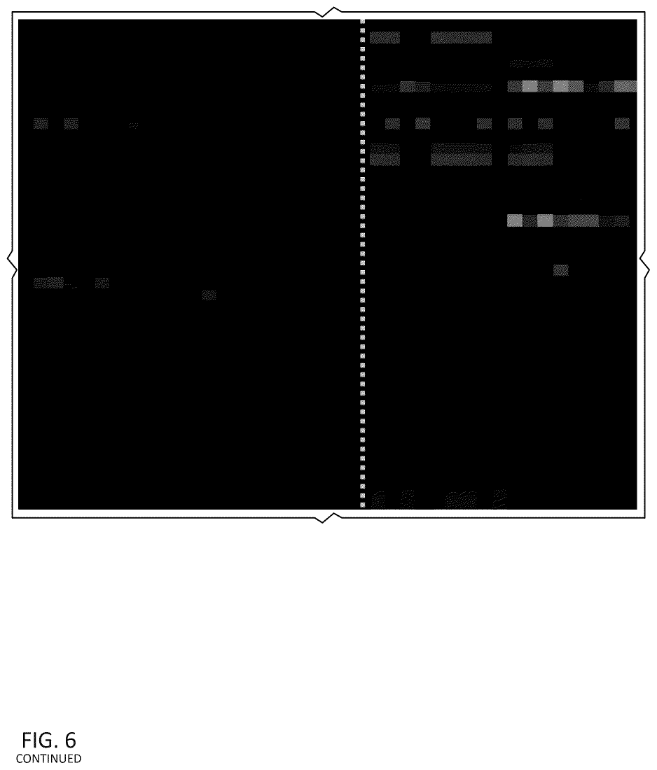

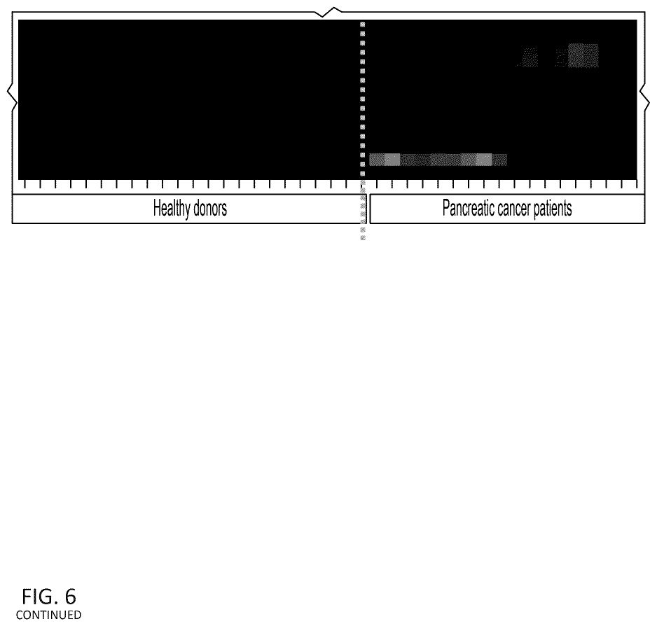

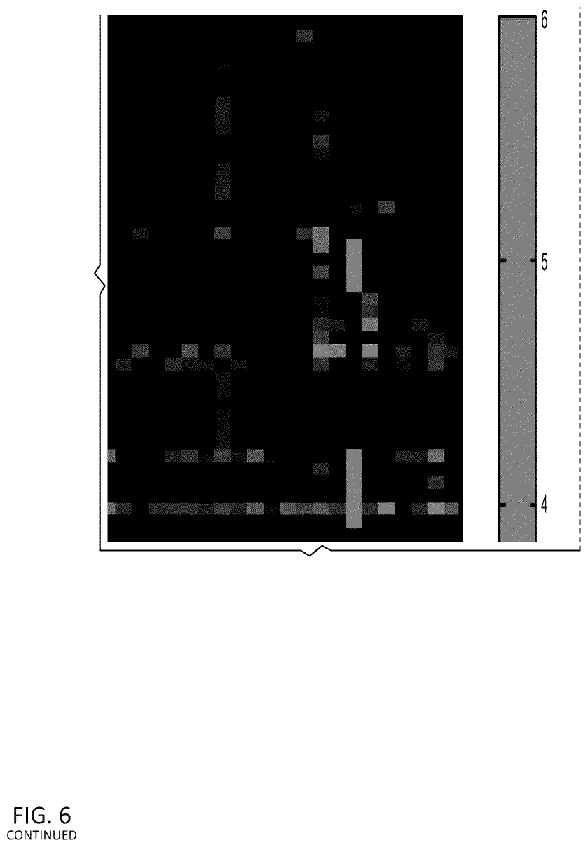

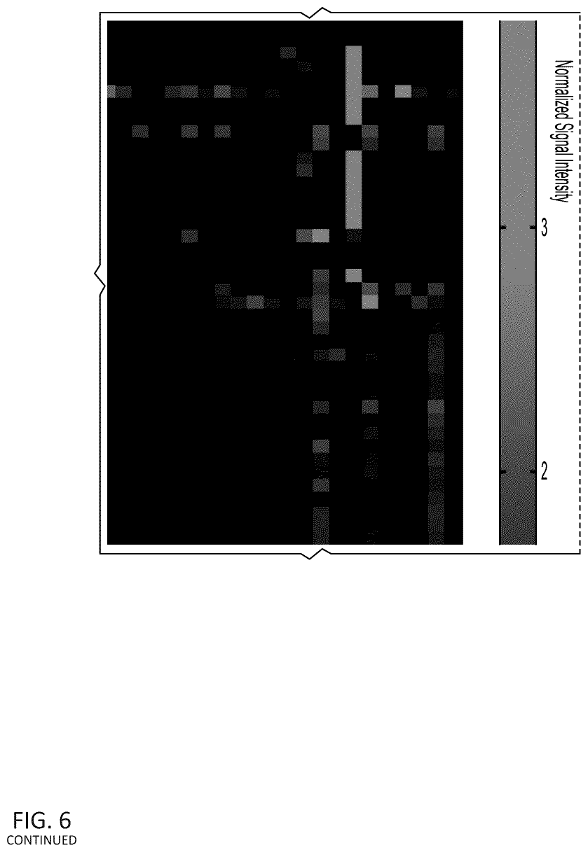

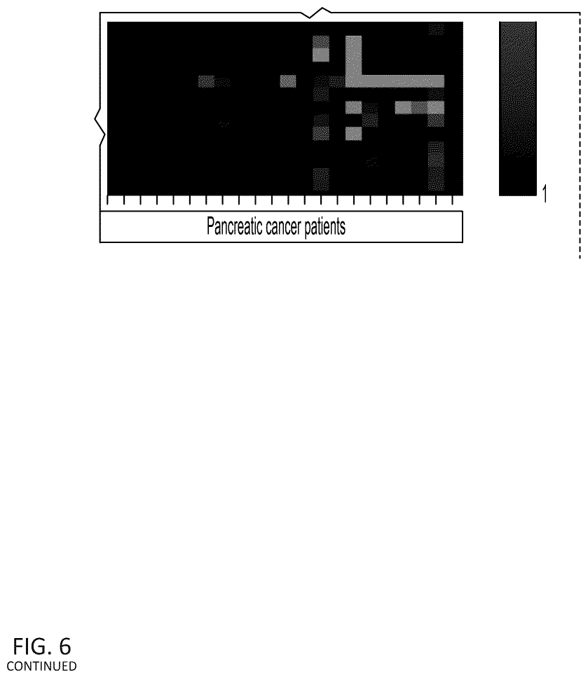

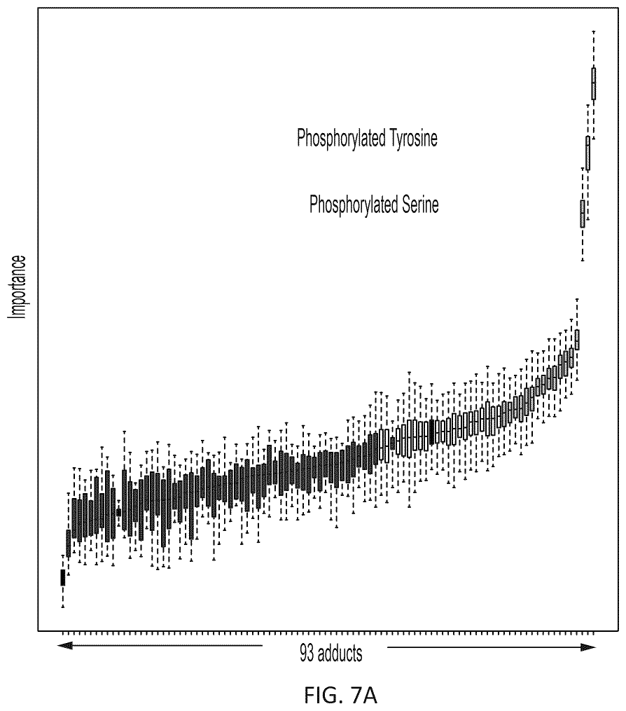

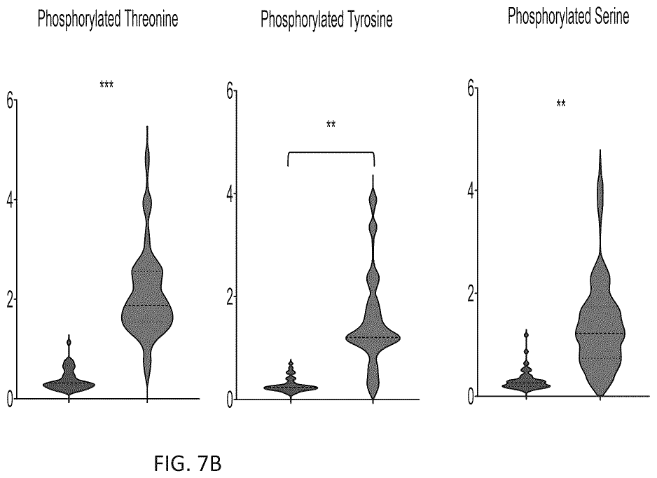

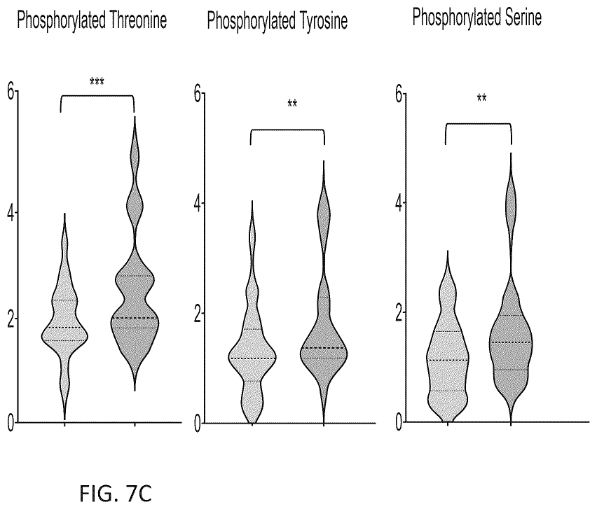

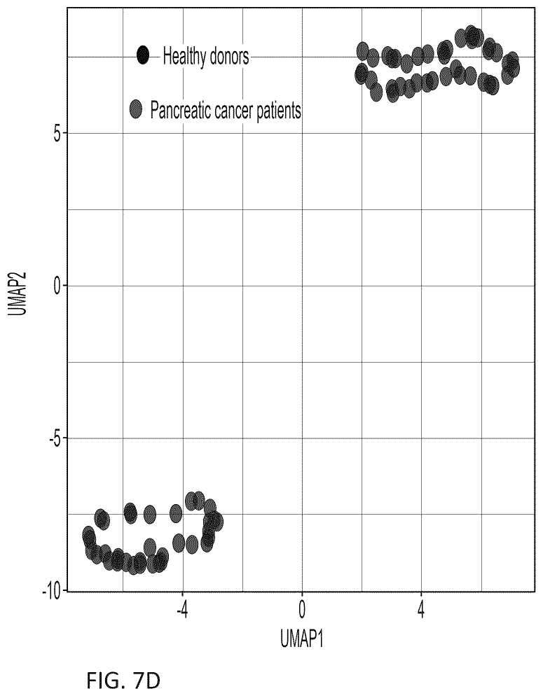

FIG. 6 provides a heatmap representation of normalized IgG reactivity to 93 adducts in Pancreatic Cancer patients (n=39) and Healthy Donors (n=40). FIG. 7: A. Boruta algorithm identified a set of 19 target adducts recognized by IgG that most efficiently discriminated between healthy donors and pancreatic cancer cases. B. IgG reactivity to the 3 phosphorylated residues as the most significant target adduct discriminating between pancreatic cancer patients (red) and healthy controls (blue)}. C. Differences in IgG reactivity between patients with (n=17—purple) or without metastasis (n=22—green). *** p-value<0.001; ** p-value<0.05 (Welch's test). D. Uniform Manifold Approximation and Projection (UMAP) comparing reactivity to the 3 phosphorylated residues between pancreatic cancer patients (red) and healthy controls (blue).

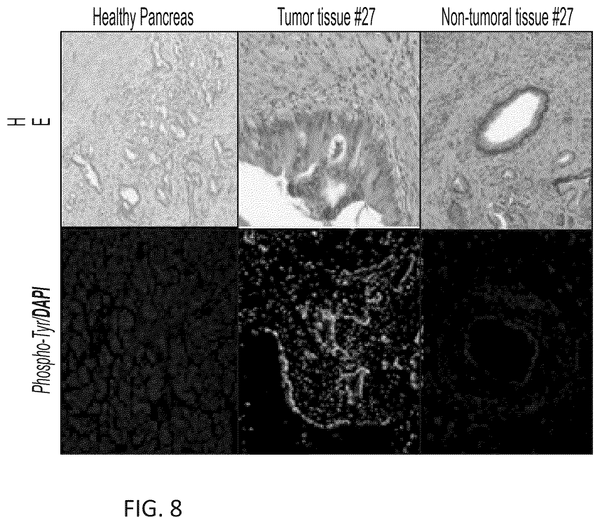

FIG. 8 provides results of detection of phosphorylated-tyrosine in tumor and adjacent normal tissue by immunofluorescence. 13 tumor/normal paired specimens were tested. 11 cases tested positive for phospho-Tyr in the tumor area and negative in the non-tumoral area. 2 cases were positive in both areas.

### Conclusions

Specific anti-adduct IgG signature in pancreatic cancer patients
Phosphorylated Threonine, Tyrosine and Serine identified as predominant targets of IgG response
Increased levels of phosphoryl groups in pancreatic tumor cells
Abnormal accumulation of phosphorylated adducts in pancreatic cancer cells triggers specific IgG response

### Example 4: Detection of Adducts Against which Antibodies from Graft Infiltrating B Cells React

B cells infiltrating cardiac grafts were isolated and used to produce 28 recombinant monoclonal antibodies. 12 recombinant monoclonal antibodies generated from blood B cells were produced and used as controls. FIG. 17 is heatmap representation showing the reactivity profile of these antibodies against the adducts set forth in Tables 1A and 1B. As is shown in FIG. 17, bilirubin is the most prominent adduct target of these antibodies.

This Example 4 and data shown in FIG. 17 demonstrate that the adduct panel of the adducts set forth in Tables 1A and 1B can be used to identify the specificity of monoclonal antibodies generated from B-cells associated with a transplanted graft. Based on this a further method embodiment is disclosed that involves exposing a sample to one or more adducts as set forth in Tables 1A and 1B, and identifying selective reactivity of the antibodies to one or more of the adducts. In this way, the specificity of the antibodies can be determined. Also, a high, selective reactivity to the one or more adducts indicates that such reactive antibodies can serve as biomarkers of immune responses or rejection of transplanted graft material.

In a further embodiment, a method is disclosed for detecting immunoreactivity against a transplanted graft material by detecting in a sample reactivity of antibodies produced against bilirubin, or any other adducts identified by the method described in the preceding paragraph. In a specific embodiment, the transplanted graft material is a transplanted organ, such as, but not limited to, a heart, lung, liver or kidney.

### REFERENCES

1. Boes M. Role of natural and immune IgM antibodies in immune responses. Molecular immunology 37, 1141-1149 (2000).
2. Lutz H U. Naturally occurring antibodies (NAbs). Springer Science+Business Media; Landes Bioscience (2012).
3. Boyden S V. Natural antibodies and the immune response. Adv Immunol 5, 1-28 (1966).
4. Holodick N E, Rodriguez-Zhurbenko N, Hernandez A M. Defining Natural Antibodies. Frontiers in immunology 8, 872 (2017).
5. Ehrenstein M R, Notley C A. The importance of natural IgM: scavenger, protector and regulator. Nature reviews Immunology 10, 778-786 (2010).
6. Chang M K, et al. Apoptotic cells with oxidation-specific epitopes are immunogenic and proinflammatory. The Journal of experimental medicine 200, 1359-1370 (2004).
7. Chen Y, Park Y B, Patel E, Silverman G J. IgM antibodies to apoptosis-associated determinants recruit C1q and enhance dendritic cell phagocytosis of apoptotic cells. J Immunol 182, 6031-6043 (2009).
8. Silverman G J. Protective natural autoantibodies to apoptotic cells: evidence of convergent selection of recurrent innate-like clones. Annals of the New York Academy of Sciences 1362, 164-175 (2015).
9. Ballow M. Primary immunodeficiency disorders: antibody deficiency. J Allergy Clin Immunol 109, 581-591 (2002).
10. Landsteiner K. The specificity of serological reactions. C. C. Thomas (1936).
11. Baumgarth N. B-1 Cell Heterogeneity and the Regulation of Natural and Antigen-Induced IgM Production. Frontiers in immunology 7, 324 (2016).
12. Tiller T, Tsuiji M, Yurasov S, Velinzon K, Nussenzweig M C, Wardemann H.

Autoreactivity in human IgG+ memory B cells. Immunity 26, 205-213 (2007).
13. Wardemann H, Nussenzweig M C. B-cell self-tolerance in humans. Adv Immunol 95, 83-110 (2007).
14. Wardemann H, Yurasov S, Schaefer A, Young J W, Meffre E, Nussenzweig M C. Predominant autoantibody production by early human B cell precursors. Science 301, 1374-1377 (2003).
15. Notkins A L. Polyreactivity of antibody molecules. Trends in immunology 25, 174-179 (2004).
16. Zhou Z H, et al. Polyreactive antibodies plus complement enhance the phagocytosis of cells made apoptotic by UV-light or HIV. Scientific reports 3, 2271 (2013).
17. Dimitrov J D, Planchais C, Roumenina L T, Vassilev T L, Kaveri S V, Lacroix-Desmazes S. Antibody polyreactivity in health and disease: statu variabilis. J Immunol 191, 993-999 (2013).
18. Avrameas S. Natural autoantibodies: from ‘horror autotoxicus’ to ‘gnothi seauton’. Immunol Today 12, 154-159 (1991).
19. Guthmiller J J, et al. Polyreactive Broadly Neutralizing B cells Are Selected to Provide Defense against Pandemic Threat Influenza Viruses. Immunity 53, 1230-1244 e1235 (2020).
20. Palmeira P, Quinello C, Silveira-Lessa A L, Zago C A, Carneiro-Sampaio M. IgG placental transfer in healthy and pathological pregnancies. Clin Dev Immunol 2012, 985646 (2012).
21. Parry G, Estelle M. Regulation of cullin-based ubiquitin ligases by the Nedd8/RUB ubiquitin-like proteins. Semin Cell Dev Biol 15, 221-229 (2004).
22. Mayer R J, Arnold J, Laszlo L, Landon M, Lowe J. Ubiquitin in health and disease. Biochimica et biophysica acta 1089, 141-157 (1991).
23. Chou M Y, et al. Oxidation-specific epitopes are dominant targets of innate natural antibodies in mice and humans. The Journal of clinical investigation 119, 1335-1349 (2009).
24. Perna A F, Ingrosso D, De Santo N G. Homocysteine and oxidative stress. Amino Acids 25, 409-417 (2003).
25. Uribarri J, et al. Advanced glycation end products in foods and a practical guide to their reduction in the diet. J Am Diet Assoc 110, 911-916 e912 (2010).
26. Perrone A, Giovino A, Benny J, Martinelli F. Advanced Glycation End Products (AGEs): Biochemistry, Signaling, Analytical Methods, and Epigenetic Effects. Oxid Med Cell Longev 2020, U.S. Pat. No. 3,818,196 (2020).
27. Ramis R, et al. How Does Pyridoxamine Inhibit the Formation of Advanced Glycation End Products? The Role of Its Primary Antioxidant Activity. Antioxidants (Basel) 8, (2019).
28. Henrick B M, et al. Bifidobacteria-mediated immune system imprinting early in life. Cell 184, 3884-3898 e3811 (2021).
29. Nguyen Q N, Himes J E, Martinez D R, Permar S R. The Impact of the Gut Microbiota on Humoral Immunity to Pathogens and Vaccination in Early Infancy. PLOS Pathog 12, e1005997 (2016).
30. Nielsen S C A, et al. Shaping of infant B cell receptor repertoires by environmental factors and infectious disease. Science translational medicine 11, (2019).
31. Olin A, et al. Stereotypic Immune System Development in Newborn Children. Cell 174, 1277-1292 e1214 (2018).
32. New J S, Dizon B L P, Fucile C F, Rosenberg A F, Kearney J F, King R G. Neonatal Exposure to Commensal-Bacteria-Derived Antigens Directs Polysaccharide-Specific B-1 B Cell Repertoire Development. Immunity 53, 172-186 e176 (2020).
33. Udemgba C, Lin A. B cells and food allergy. Current opinion in pediatrics, (2021).
34. Schwarz A, et al. IgG and IgG4 to 91 allergenic molecules in early childhood by route of exposure and current and future IgE sensitization: Results from the Multicentre Allergy Study birth cohort. J Allergy Clin Immunol, (2016).
35. Cheung P, et al. Single-Cell Chromatin Modification Profiling Reveals Increased Epigenetic Variations with Aging. Cell 173, 1385-1397 e1314 (2018).
36. Horvath S, et al. DNA methylation age analysis of rapamycin in common marmosets. Geroscience, (2021).
37. Lehallier B, et al. Undulating changes in human plasma proteome profiles across the lifespan. Nature medicine 25, 1843-1850 (2019).
38. Amara K, et al. Monoclonal IgG antibodies generated from joint-derived B cells of RA patients have a strong bias toward citrullinated autoantigen recognition. The Journal of experimental medicine 210, 445-455 (2013).
39. Volkov M, van Schie K A, van der Woude D. Autoantibodies and B Cells: The ABC of rheumatoid arthritis pathophysiology. Immunol Rev 294, 148-163 (2020).
40. Steen J, et al. Recognition of Amino Acid Motifs, Rather Than Specific Proteins, by Human Plasma Cell-Derived Monoclonal Antibodies to Posttranslationally Modified Proteins in Rheumatoid Arthritis. Arthritis Rheumatol 71, 196-209 (2019).
41. Kim S J, Gershov D, Ma X, Brot N, Elkon K B. I-PLA (2) activation during apoptosis promotes the exposure of membrane lysophosphatidylcholine leading to binding by natural immunoglobulin M antibodies and complement activation. The Journal of experimental medicine 196, 655-665 (2002).
42. Utz P J, Hottelet M, Schur P H, Anderson P. Proteins phosphorylated during stress-induced apoptosis are common targets for autoantibody production in patients with systemic lupus erythematosus. The Journal of experimental medicine 185, 843-854 (1997).
43. Binder C J, Silverman G J. Natural antibodies and the autoimmunity of atherosclerosis. Springer seminars in immunopathology 26, 385-404 (2005).
44. Milner E C, Anolik J, Cappione A, Sanz I. Human innate B cells: a link between host defense and autoimmunity? Springer seminars in immunopathology 26, 433-452 (2005).
45. Martin F, Kearney J F. B1 cells: similarities and differences with other B cell subsets. Current opinion in immunology 13, 195-201 (2001).
46. Zorn E, See S B. Polyreactive natural antibodies in transplantation. Current opinion in organ transplantation 22, 8-13 (2017).
47. Zhou Z H, Tzioufas A G, Notkins A L. Properties and function of polyreactive antibodies and polyreactive antigen-binding B cells. J Autoimmun 29, 219-228 (2007).
48. Weller S, et al. Human blood IgM “memory” B cells are circulating splenic marginal zone B cells harboring a prediversified immunoglobulin repertoire. Blood 104, 3647-3654 (2004).
49. Porcheray F, et al. Expansion of Polyreactive B Cells Cross-Reactive to HLA and Self in the Blood of a Patient With Kidney Graft Rejection. Am J Transplant 12, 2088-2097 (2012).
50. Porcheray F, et al. Polyreactive antibodies developing amidst humoral rejection of human kidney grafts bind apoptotic cells and activate complement. Am J Transplant 13, 2590-2600 (2013).
51. Porcheray F, et al. Chronic humoral rejection of human kidney allografts associates with broad autoantibody responses. Transplantation 89, 1239-1246 (2010).

1. A method for detecting the presence of natural antibodies reactive to one or more adducts in one or more subjects comprising,
analyzing a biological sample provided from said one or more subjects for the presence or absence of natural antibodies reactive to one or more adducts as set forth in Tables 1A and 1B, wherein the natural antibodies optionally are IgG antibodies and/or IgM antibodies.

2. The method according to claim 1, wherein the biological sample from the subject is selected from blood, tissue or fluid, preferably selected from hair, skin, nails, saliva, synovia, urine, liquor and blood, or wherein the biological sample is cord blood or peripheral blood.

3. The method according to claim 1, wherein the analyzing is performed using an immunoassay, preferably with direct or indirect coupling of one reactant to a detectable moiety, wherein, optionally, wherein the detectable moiety is an antibody that binds to natural antibodies of a species to which the subject belongs conjugated or associated with a fluorometric marker.

4. The method according to claim 3 wherein analyzing is carried out using an ELISA, RIA, multiplex immunoassay or immunofluorescence assay, western blot, line assay, dot blot assay.

5. The method according to claim 4, wherein analyzing comprises subjecting the biological sample to a solid support comprising at least one adduct as set forth in Tables 1A and Table 1B bound to a surface thereof.

6. The method of claim 5, further comprising washing the wells, and subjecting the washed wells to one or more reagents that bind to a natural antibody and produces a detectable signal.

7. The method of claim 6, wherein the one or more reagents comprise an antibody that selectively binds to natural antibodies of a species to which the subject belongs, wherein the antibody that selectively binds to natural antibodies of a species to which the subject belongs optionally is conjugated or associated with a fluorometric or colorimetric marker.

8. The method of claim 6, further comprising comparing the detectable signal of the natural antibody with a control.

9. A multiplexed assay method capable of measuring the interaction of one or more natural antibodies in a biological sample with one or more solid supports each comprising a solid phase surface comprising: contacting the solid supports with the biological sample, the solid phase surfaces comprising one or more individual chemical adducts selected from Tables 1A and 1B, and determining the level of natural antibody captured by the adducts on the solid phase surfaces.

10. A multiplexed assay method of claim 9, wherein the solid supports comprise beads or microspheres, and the method further comprises
mixing a biological sample with the solid supports to form a mixture; washing the mixture to remove unbound biological sample from the solid supports; and detecting one or more natural antibodies from the biological sample that are captured by an adduct or multiple adducts using a fluorescently labeled reagent that binds to the one or more natural antibodies.

11. A kit comprising a multiplex reagent, the multiplex reagent comprising a plurality of microspheres attached, directly or indirectly, to one or more adducts set forth in Table 1B, wherein the plurality of microspheres attached to an individual adduct comprise a distinct fluorometric signature.

12. A method for determining whether a subject has pancreatic cancer, the method comprising analyzing a biological sample from the subject for presence of (i) anti-phosphorylated threonine antibodies, (ii) anti-phosphorylated tyrosine antibodies and (iii) anti-phosphorylated serine antibodies, and diagnosing a patient as having pancreatic cancer or risk of pancreatic cancer if the biological sample comprises (i), (ii) and (iii), or elevated levels of (i), (ii) or (iii) relative to a healthy control, and optionally administering a cancer therapy to the subject if diagnosed to have pancreatic cancer.

13. A method of identifying specificity of antibodies against a transplanted graft material in a subject, the method comprising:
exposing a sample from the subject to one or more adducts as set forth in Tables 1A and 1B; and
determining reactivity of antibodies in the sample to one or more of the adducts.

14. The method of claim 13, wherein the transplanted graft material comprises a transplanted organ.

15. The method of claim 13, further comprising identifying an adduct to which the one or more antibodies reacts as a biomarker for an immune response against the transplanted graft material.

16. The method of claim 15, wherein the biomarker comprises one or more of methylated proteins, phosphorylated proteins, or nucleotide derivatives.

17. The method of claim 16, wherein the biomarker is selected from bilirubin, MDA-lysine, ADP-ribose, trimethylated-lysine and phosphorylated-serine.

18. The method of claim 13, further comprising administering an effective amount of an immunosuppressive agent to the subject if reactivity is detected.

19. A kit for use in a method for determining an immune response or level of immune response against transplanted graft material, the kit comprising a solid support having a surface that is treated with a biomarker selected from methylated proteins, phosphorylated proteins, and/or several nucleotide derivatives, wherein, optionally, the solid support comprises a multiwell plate, a bead, or a microsphere, and wherein the kit optionally further comprises a detection reagent.

20. The kit of claim 19, wherein the biomarker is selected from bilirubin, MDA-lysine, ADP-ribose, trimethylated-lysine or phosphorylated-serine, or a combination thereof.
